SEQUENCE LISTING (4 sequences)

SEQ ID NO: 1 - DNA, 3706 bp, Burkholderia cepacia
  source 1..3706: mol_type = genomic DNA
  CDS 258..761: protein_id = 2; translation = MHNDNTPHSRRHGDAAASGITRRQWLQGALALTAAGLTGSLTLR ALADNPGTAPLDTFMTLSESLTGKKGLSRVIGERLLQALQKGSFKTADSLPQLAGALA SGSLTPEQESLALTILEAWYLGIVDNVVITYEEALMFGVVSDTLVIRSYCPNKPGFWA DKPIERQA
  CDS 764..2380: protein_id = 3; translation = MADTDTQKADVVVVGSGVAGAIVAHQLAMAGKAVILLEAGPRMP RWEIVERFRNQPDKMDFMAPYPSSPWAPHPEYGPPNDYLILKGEHKFNSQYIRAVGGT TWHWAASAWRFIPNDFKMKSVYGVGRDWPIQYDDLEPYYQRAEEELGVWGPGPEEDLY SPRKQPYPMPPLPLSFNEQTIKTALNNYDPKFHVVTEPVARNSRPYDGRPTCCGNNNC MPICPIGAMYNGIVHVEKAERAGAKLIENAVVYKLETGPDKRIVAALYKDKTGAEHRV EGKYFVLAANGIETPKILLMSANRDFPNGVANSSDMVGRNLMDHPGTGVSFYASEKLW PGRGPQEMTSLIGFRDGPFRATEAAKKIHLSNLSRIDQETQKIFKAGKLMKPDELDAQ IRDRSARYVQFDCFHEILPQPENRIVPSKTATDAIGIPRPEITYAIDDYVKRGAAHTR EVYATAAKVLGGTDVVFNDEFAPNNHITGSTIMGADARDSVVDKDCRTFDHPNLFISS SATMPTVGTVNVTLTIAALALRMSDTLKKEV
  CDS 2386..3660: protein_id = 4; translation = VRKSTLTFLIAGCLALPGFARAADAADPALVKRGEYLATAGDCM ACHTVKGGKPYAGGLGMPVPMLGKIYTSNITPDPDTGIGKWTFEDFERAVRHGVSKNG DNLYPAMPYVSYAKITDDDVRALYAYFMHGVEPVKQAPPKNEIPALLSMRWPLKIWNW LFLKDGPYQPKPSQSAEWNRGAYLVQGLAHCSTCHTPRGIAMQEKSLDETGGSFLAGS VLAGWDGYNITSDPNAGIGSWTQQQLVQYLRTGSVPGVAQAAGPMAEAVEHSFSKMTE ADIGAIATYVRTVPAVADSNAKQPRSSWGKPAEDGLKLRGVALASSGIDPARLYLGNC ATCHQMQGKGTPDGYYPSLFHNSTVGASNPSNLVQVILNGVQRKIGSEDIGMPAFRYD LNDAQIAALTNYVTAQFGNPAAKVTEQDVAKLR
aagctttctgtttgattgcacgcgattctaaccgagcgtctgtgaggcggaacgcgacatgcttcgtgtcgcacacgtgtcgcgccgacgacacaaaaatgcagcgaaatggctgatcgttacgaatggctgacacattgaatggactataaaaccattgtccgttccggaatgtgcgcgtacatttcaggtccgcgccgatttttgagaaatatcaagcgtggttttcccgaatccggtgttcgagagaaggaaacatgcacaacgacaacactccccactcgcgtcgccacggcgacgcagccgcatcaggcatcacgcggcgtcaatggttgcaaggcgcgctggcgctgaccgcagcgggcctcacgggttcgctgacattgcgggcgcttgcagacaaccccggcactgcgccgctcgatacgttcatgacgctttccgaatcgctgaccggcaagaaagggctcagccgcgtgatcggcgagcgcctgctgcaggcgctgcagaagggctcgttcaagacggccgacagcctgccgcagctcgccggcgcgctcgcgtccggttcgctgacgcctgaacaggaatcgctcgcactgacgatcctcgaggcctggtatctcggcatcgtcgacaacgtcgtgattacgtacgaggaagcattaatgttcggcgtcgtgtccgatacgctcgtgatccgttcgtattgccccaacaaacccggcttctgggccgacaaaccgatcgagaggcaagcctgatggccgataccgatacgcaaaaggccgacgtcgtcgtcgttggatcgggtgtcgcgggcgcgatcgtcgcgcatcagctcgcgatggcgggcaaggcggtgatcctgctcgaagcgggcccgcgcatgccgcgctgggaaatcgtcgagcgcttccgcaatcagcccgacaagatggacttcatggcgccgtacccgtcgagcccctgggcgccgcatcccgagtacggcccgccgaacgactacctgatcctgaagggcgagcacaagttcaactcgcagtacatccgcgcggtgggcggcacgacgtggcactgggccgcgtcggcgtggcgcttcattccgaacgacttcaagatgaagagcgtgtacggcgtcggccgcgactggccgatccagtacgacgatctcgagccgtactatcagcgcgcggaggaagagctcggcgtgtggggcccgggccccgaggaagatctgtactcgccgcgcaagcagccgtatccgatgccgccgctgccgttgtcgttcaacgagcagaccatcaagacggcgctgaacaactacgatccgaagttccatgtcgtgaccgagccggtcgcgcgcaacagccgcccgtacgacggccgcccgacttgttgcggcaacaacaactgcatgccgatctgcccgatcggcgcgatgtacaacggcatcgtgcacgtcgagaaggccgaacgcgccggcgcgaagctgatcgagaacgcggtcgtctacaagctcgagacgggcccggacaagcgcatcgtcgcggcgctctacaaggacaagacgggcgccgagcatcgcgtcgaaggcaagtatttcgtgctcgccgcgaacggcatcgagacgccgaagatcctgctgatgtccgcgaaccgcgatttcccgaacggtgtcgcgaacagctcggacatggtcggccgcaacctgatggaccatccgggcaccggcgtgtcgttctatgcgagcgagaagctgtggccgggccgcggcccgcaggagatgacgtcgctgatcggtttccgcgacggtccgttccgcgcgaccgaagcggcgaagaagatccacctgtcgaacctgtcgcgcatcgaccaggagacgcagaagatcttcaaggccggcaagctgatgaagcccgacgagctcgacgcgcagatccgcgaccgttccgcacgctacgtgcagttcgactgcttccacgaaatcctgccgcaacccgagaaccgcatcgtgccgagcaagacggcgaccgatgcgatcggcattccgcgccccgagatcacgtatgcgatcgacgactacgtgaagcgcggcgccgcgcatacgcgcgaggtctacgcgaccgccgcgaaggtgctcggcggcacggacgtcgtgttcaacgacgaattcgcgccgaacaatcacatcacgggctcgacgatcatgggcgccgatgcgcgcgactccgtcgtcgacaaggactgccgcacgttcgaccatccgaacctgttcatttcgagcagcgcgacgatgccgaccgtcggtaccgtaaacgtgacgctgacgatcgccgcgctcgcgctgcggatgtcggacacgctgaagaaggaagtctgaccgtgcggaaatctactctcactttcctcatcgccggctgcctcgcgttgccgggcttcgcgcgcgcggccgatgcggccgatccggcgctggtcaagcgcggcgaatacctcgcgaccgccggcgactgcatggcctgccacaccgtgaagggcggcaagccgtacgcgggcggccttggcatgccggtaccgatgctcggcaagatctacacgagcaacatcacgcccgatcccgatacgggcatcggcaaatggacgttcgaggacttcgagcgcgcggtgcggcacggcgtgtcgaagaacggcgacaacctgtatccggcgatgccgtacgtgtcgtacgcgaagatcacggacgacgacgtacgcgcgctgtacgcctacttcatgcacggcgtcgagccggtcaagcaggcgccgccgaagaacgagattcccgcgctgctcagcatgcgctggccgctgaagatctggaactggctgttcctgaaggacggcccgtaccagccgaagccgtcgcagagcgccgaatggaatcgcggcgcgtatctggtgcagggtctcgcgcactgcagcacgtgccacacgccgcgcggcatcgcgatgcaggagaagtcgctcgacgaaaccggcggcagcttcctcgcggggtcggtgctcgccggctgggacggctacaacatcacgtcggacccgaatgcggggatcggcagctggacgcagcagcagctcgtgcagtatttgcgcaccggcagcgtgccgggcgtcgcgcaggcggccgggccgatggccgaggcggtcgagcacagcttctcgaagatgaccgaagcggacatcggtgcgatcgccacgtacgtccgcacggtgccggccgttgccgacagcaacgcgaagcagccgcggtcgtcgtggggcaagccggccgaggacgggctgaagctgcgcggtgtcgcgctcgcgtcgtcgggcatcgatccggcgcggctgtatctcggcaactgcgcgacgtgccaccagatgcagggcaagggcacgccggacggctattacccgtcgctgttccacaactccaccgtcggcgcgtcgaatccgtcgaacctcgtgcaggtgatcctgaacggcgtgcagcgcaagatcggcagcgaggatatcgggatgcccgctttccgctacgatctgaacgacgcgcagatcgccgcgctgacgaactacgtgaccgcgcagttcggcaatccggcggcgaaggtgacggagcaggacgtcgcgaagctgcgctgacatagtcgggcgcgccgacacggcgcaaccgataggacaggag

SEQ ID NO: 2 - protein, 168 aa, Burkholderia cepacia
MHNDNTPHSRRHGDAAASGITRRQWLQGALALTAAGLTGSLTLRALADNPGTAPLDTFMTLSESLTGKKGLSRVIGERLLQALQKGSFKTADSLPQLAGALASGSLTPEQESLALTILEAWYLGIVDNVVITYEEALMFGVVSDTLVIRSYCPNKPGFWADKPIERQA

SEQ ID NO: 3 - protein, 539 aa, Burkholderia cepacia
MADTDTQKADVVVVGSGVAGAIVAHQLAMAGKAVILLEAGPRMPRWEIVERFRNQPDKMDFMAPYPSSPWAPHPEYGPPNDYLILKGEHKFNSQYIRAVGGTTWHWAASAWRFIPNDFKMKSVYGVGRDWPIQYDDLEPYYQRAEEELGVWGPGPEEDLYSPRKQPYPMPPLPLSFNEQTIKTALNNYDPKFHVVTEPVARNSRPYDGRPTCCGNNNCMPICPIGAMYNGIVHVEKAERAGAKLIENAVVYKLETGPDKRIVAALYKDKTGAEHRVEGKYFVLAANGIETPKILLMSANRDFPNGVANSSDMVGRNLMDHPGTGVSFYASEKLWPGRGPQEMTSLIGFRDGPFRATEAAKKIHLSNLSRIDQETQKIFKAGKLMKPDELDAQIRDRSARYVQFDCFHEILPQPENRIVPSKTATDAIGIPRPEITYAIDDYVKRGAAHTREVYATAAKVLGGTDVVFNDEFAPNNHITGSTIMGADARDSVVDKDCRTFDHPNLFISSSATMPTVGTVNVTLTIAALALRMSDTLKKEV

SEQ ID NO: 4 - protein, 425 aa, Burkholderia cepacia
VRKSTLTFLIAGCLALPGFARAADAADPALVKRGEYLATAGDCMACHTVKGGKPYAGGLGMPVPMLGKIYTSNITPDPDTGIGKWTFEDFERAVRHGVSKNGDNLYPAMPYVSYAKITDDDVRALYAYFMHGVEPVKQAPPKNEIPALLSMRWPLKIWNWLFLKDGPYQPKPSQSAEWNRGAYLVQGLAHCSTCHTPRGIAMQEKSLDETGGSFLAGSVLAGWDGYNITSDPNAGIGSWTQQQLVQYLRTGSVPGVAQAAGPMAEAVEHSFSKMTEADIGAIATYVRTVPAVADSNAKQPRSSWGKPAEDGLKLRGVALASSGIDPARLYLGNCATCHQMQGKGTPDGYYPSLFHNSTVGASNPSNLVQVILNGVQRKIGSEDIGMPAFRYDLNDAQIAALTNYVTAQFGNPAAKVTEQDVAKLR

(19) United States
(12) Patent Application Publication
(10) Pub. No.: US 2026/0242841 A1
(43) Pub. Date: Aug. 20, 2026

(54) DIRECT ELECTRON TRANSFER TYPE ENZYME EMPLOYING EXTENDED GATE FIELD EFFECT TRANSISTOR BIOSENSORS

(71) Applicants: THE UNIVERSITY OF NORTH CAROLINA AT CHAPEL HILL, Chapel Hill, NC (US); ARKRAY, INC., Kyoto-shi, Kyoto (JP)

(72) Inventors: Koji SODE, Chapel Hill, NC (US); Mika HATADA, Chapel Hill, NC (US); David PROBST, Chapel Hill, NC (US)

(73) Assignees: THE UNIVERSITY OF NORTH CAROLINA AT CHAPEL HILL, Chapel Hill, NC (US); ARKRAY, INC., Kyoto-shi, Kyoto (JP)

(21) Appl. No.: 19/489,514

(22) PCT Filed: May 31, 2024
(86) PCT No.: PCT/US2024/032015

§ 371 Date: Dec. 2, 2025

Related U.S. Application Data
(60) Provisional application No. 63/505,824, filed on Jun. 2, 2023.

(51) Int. Cl. C12Q 1/00; G01N 27/327; G01N 27/414

(52) U.S. Cl. CPC C12Q 1/005; C12Q 1/006; G01N 27/3271; G01N 27/3276; G01N 27/4145

(57) ABSTRACT

The present disclosure relates to a biosensor comprising an enzyme electrode comprising a direct electron transfer type oxidoreductase wherein said enzyme electrode is electrically connected to a gate electrode of a field effect transistor (FET), and a method of measuring a substance using the biosensor.

### FIELD OF THE INVENTION

The invention relates to a biosensor such as glucose sensor and lactate sensor wherein DET-type oxidoreductase-immobilized enzyme electrode is connected to a gate of EG-FET.

### BACKGROUND OF THE INVENTION

The development of point of care testing (POCT) and continuous glucose sensors (CGM's) has been vastly studied using novel electrochemical architectures, enzyme engineering, and algorithms¹⁻². Traditionally using glucose oxidase/dehydrogenase and amperometric electrochemistry to measure glucose based on either the reduction/oxidation of a product or reactant. Limitations exist using these traditional methods, include dependence of electrode surface area, requiring sufficient counter electrode surface area, and challenges with electroactive interference. To overcome these obstacles both researchers and industry has developed many innovations, including using polymers to prevent interfering substrates from reaching the electrode surface; engineering novel enzymes which use lower potentials than the oxidation/reduction potential of interference, or employing new algorithms to eliminate the background noise caused by these substrate³⁻⁶. Other challenges, such as electrode surface area, and minimization has been tackled through developing new electrochemical sensing approaches such as open circuit potential (OCP). OCP measured the change in an electrode's potential compared to a stable reference (usually Ag/AgCl) based on the Nernst equation. This method has been shown to be both more stable than traditional amperometry, and that the signal is independent to the electrodes surface area⁷⁻⁹. Our group has previously demonstrated that when measuring a junction potential, or junction potential driven change, that the signal is equivalent over many different surface areas. Other methods for the detection of glucose have included field effect transistors (FET's) and extended gate FET's (EGFETs) which measure a change in either voltage, current or impedance depending on how the sensor is laid out and which architecture the groups is using. Several groups have developed FET type glucose sensors using glucose oxidase (GOX) or nonenzymatic glucose detection¹⁰⁻¹³. Most enzyme free approaches using FET take advantage of iron oxide, or zinc oxide which under the proper environment ca directly oxidize glucose, albeit with uncertain specificity compared to other electroactive interfering substrates that exist in the body¹⁴. Recently the use of EGFET's have been employed to measure glucose, but with similar methods, such as using copper oxide for enzyme free glucose detection¹⁵. This approach will still suffer from nonspecific interactions and is highly sensitive to pH of the local environment, which will fluctuate due to the production of gluconic acid. To the best of our knowledge the use of direct electron transfer type enzyme (DET) has yet to be characterized for glucose detection using an EGFET. Our group has previously reported about the use of DET type FAD glucose dehydrogenase (DET-FADGDH) for glucose sensing using OCP, amperometry, and impedance^{7, 16, 17}. Investigation using EGFET is still not performed, and may pose opportunities for better sensitivity, stability and miniaturization compared to conventional sensors that rely on OCP changes.

Lactate measurement has generally been performed using conventional amperometric detection, where either lactate oxidase (Lox) or lactate dehydrogenase (LDH) is immobilized onto an electrode surface. Upon interacting with lactate, the enzyme transfers an electron to either oxygen-forming hydrogen peroxide (Lox pathway) or to an external mediator (LDH pathway). After this reaction the byproduct is then oxidized at the working electrode, which is held at a sufficient overpotential. While convenient and simple to design, current Lox- and LDH-based amperometric lactate sensors possess several inherent drawbacks. The potential necessary to oxidize byproducts such as hydrogen peroxide can exceed 0.4 V vs. Ag/AgCl, leading to nonspecific oxidation at the electrode surface when testing samples taken from patients¹⁸⁻²¹. These may include species such as ascorbic acid and acetaminophen, which are quickly oxidized at the working electrode surface when overpotentials above 0.4 V vs. Ag/AgCl are applied. Additionally, amperometric sensing becomes increasingly difficult as the size of the electrodes used is decreased. Based on the Cottrell equation, measured current is proportional to the electrode surface area, rendering detection at smaller electrodes more challenging as the signals involved become ever smaller and more sensitive to sources of noise. The use of nanotechnology has been a key method to improve on this limitation using methods that increase the Debye volume, apparent surface area²². However the ultimate reliance of these sensors on current measurement means that limitations imposed by the working electrode surface area cannot be fully mitigated²³.

Various innovations have been developed to overcome these obstacles, focusing on direct engineering of the utilized enzymes, as well as on novel measurement modalities and architectures used to detect lactate^{28, 24-27} These approaches have included the creation of novel enzymes which mitigate signals due to interferent species by lowering the required overpotential or eliminating the need for external electron mediators such as oxygen. One such example was demonstrated by Sode et al., who predicted the pathway of oxygen through Aerococcus viridans Lox (AvLOx), and performed a point mutation to mitigate all oxygenase activity while retaining dehydrogenase activity²⁹. Other approaches for improving sensor performance have involved conjugating novel electron mediators directly to the enzyme. This strategy was previously demonstrated by Sode et al., whose group conjugated amine-reactive phenazine ethosulfate (arPES) to glucose dehydrogenase²⁴. This modification lowered the oxidation overpotential of the sensor to 0.0 V vs. Ag/AgCl, mitigating signals arising from potential in vivo interferent species such as ascorbic acid, uric acid, and acetaminophen. Our group has previously described the use of arPES-modified lactate dehydrogenase for lactate sensing using amperometry and OCP^{24,30}.

To the best of our knowledge, an EGFET lactate sensor employing quasi-direct electron transfer between a modified mediator and the extended gate electrode has not yet been reported.

### SUMMARY OF THE INVENTION

This invention discloses the biosensors and their principles employing direct electron transfer (DET)-type oxidoreductases (enzymes) as biosensing molecules, and extended gate effect transistor (EGFET) as a transducer, where DET-type enzyme is immobilized on the surface of electrode connected with the gate of EGFET.

One aspect of the invention is to provide a biosensor comprising an enzyme electrode comprising a direct electron transfer type oxidoreductase wherein said enzyme electrode is electrically connected to a gate electrode of a field effect transistor (FET).

In one embodiment, said direct electron transfer type oxidoreductase comprises an electron transfer subunit and/or electron transfer domain.

In one embodiment, said direct electron transfer type oxidoreductase comprises a cytochrome containing subunit.

In one embodiment, said direct electron transfer type oxidoreductase is selected from the group consisting of glucose dehydrogenase, lactate dehydrogenase, sorbitol dehydrogenase, D-fructose dehydrogenase, D-glucoside-3-dehydrogenase, and cellobiose dehydrogenase.

In one embodiment, said direct electron transfer type oxidoreductase is an oxidoreductase which is modified with an electron acceptor.

In one embodiment, said electron acceptor is selected from the group consisting of 1,4-naphthoquinone, vitamin K3, 9,10-phenanthrenequinone, 1,2-naphthoquinone, p-xyloquinone, methylbenzoquinone, 2,6-dimethylbenzoquinone, sodium 1,2-naphthoquinone-4-sulfonate, 1,4-anthraquinone, tetramethylbenzoquinone, and thymoquinone), phenylenediamine compounds (for example, N,N-dimethyl-1,4-phenylenediamine and N,N,N′,N′-tetramethyl-1,4-phenylenediamine), 1-methoxy-PMS (1-methoxy-5-methylphenazinium methylsulfate), PES (phenazine ethosulfate), coenzyme Q0, AZURE A chloride, phenosafranin, 6-aminoquinoxaline, and tetrathiafulvalene.

In one embodiment, said biosensor is a glucose sensor or a lactate sensor.

In one embodiment, said biosensor further comprising a reference electrode.

Another aspect of the invention is to provide a measuring method comprising bringing a sample containing a measuring target substance into contact with the biosensor as described above and measuring a signal generated via a reaction between the substance and the oxidoreductase.

In one embodiment, the measuring target substance is glucose or lactate.

In one embodiment, the sample is selected from a blood sample, a sweat sample, an interstitial fluid sample or a saliva sample.

In one embodiment, a current flowing between the source electrode and the drain electrode of the transistor is measured.

In one embodiment, the measuring step is conducted while applying an electric potential of V between the source and the drain.

### BRIEF DESCRIPTION OF DRAWINGS

FIG. 1: Scheme of the biosensor in one embodiment

FIG. 2: A: I_{DS} dependency on V_{GS} and glucose concentration. The inset figure zooms into the 0.8-1V range for better depict the change in current upon glucose addition. B: Calibration curve derived with glucose addition while holding the V_{GS} at 1V. C: Impact of shifting the V_{DS} from 0.5V down to 0.0V. The relative change with glucose was comparable for both settings.

FIG. 3: Calibration curves for glucose using difference solution compositions (buffer, artificial sweat, and artificial serum). The inset is the detection of glucose range from 10 μM to 90 μM which is more correlative to sweat concentrations.

FIG. 4: Glucose detection in a continuous platform using a constant voltage between the gate and source electrode of 1V in artificial sweat. The inset is the calculated calibration curve based on the continuous detection using 3 replicates.

FIG. 5: Glucose detection in a continuous platform using a constant voltage between the gate and source electrode of 1V in artificial ISF. The inset is the calculated calibration curve based on the continuous detection using 3 replicates.

FIG. 6: A: Signal change upon addition of various interference substrates. B: Reversibility and signal retention of 20 mM and 0 glucose using artificial sweat. C: Stability over 24 hours in artificial sweat with 20 mM glucose. D: Stability over 24 hours in artificial ISF with 20 mM glucose.

FIG. 7: A: Current measured through the FET drain and source during a linear sweep (0.0 V-1.5 V) of the potential applied to the reference electrode (V_{Ref}) at various lactate concentrations of 0-20 mM. B: Magnified view of 2A showing the region in which V_{Ref} is swept from 1.45 V-1.55 V. C: Calibration curve showing the relationship between lactate addition and I_{D} (n=3 replicates) with V_{Ref} held constant at 1.5 V (with V_{DS} also held constant at 0.5 V).

FIG. 8: A: Example response during continuous lactate detection in artificial sweat, while sequentially adding lactate to generate final concentrations from 0 mM-20 mM. B: Calibration curve generated from continuous lactate sensing, showing Alp at various lactate concentrations using the blank to normalize. Error bars are standard deviation calculated using n=3 replicates. C: Representative response to 3 mM lactate with, or without interferent species present (n=3). D: Representative response of the arPES-modified AvLOx sensor over 12 hours while maintaining a constant lactate concentration (20 mM) in artificial sweat.

FIG. 9: The performance of the microwire type sensor in human plasma measuring either a) OCP, or b) I_{D}. V_{ref} was swept from −1.5 V to −0.5 V. c) Calibration curve for OCP 60 seconds after amperometric pulse, d) I_{D} calibration curve.

FIG. 10: Calibration of human serum samples using disposable glucometer, validating measurements from EGFET glucose sensor.

FIG. 11: Percent change of I_{D} (upper) when using a junction reference electrode versus a pseudo-reference electrode (wire) measuring using the EGFET, and OCP (bottom).

### DETAILED DESCRIPTION

A biosensor according to one embodiment of the invention comprises an enzyme electrode comprising a direct electron transfer type oxidoreductase wherein said enzyme electrode is electrically connected to a gate electrode of a field effect transistor.

The FET comprises a source electrode and a drain electrode as well as a drain electrode. More specifically, FET comprises a semiconductor layer; a source electrode and a drain electrode provided on the semiconductor layer; and a gate electrode separated from the semiconductor layer by an insulating layer. The FET is preferably a MOS-type.

In the biosensor, an enzyme electrode is electrically connected to the gate electrode of the FET.

The enzyme electrode is placed in a reaction chamber such as an electrochemical cell and can react with an enzyme substrate upon addition of a sample containing the enzyme substrate. The biosensor preferably comprises a reference electrode which is also placed in the reaction chamber.

### <Enzyme Electrode>

The enzyme electrode comprises an oxidoreductase disposed on an electrode material.

The electrode can be formed using a metallic material or a carbon material, wherein examples of the metallic material include gold (Au), platinum (Pt), silver (Ag), and palladium (Pd), and examples of the carbon material include carbons such as graphite, carbon nanotube, graphene, and mesoporous carbon. The electrode may be provided on an insulating substrate formed with an insulating material, wherein examples of the insulating material include resins (plastics) such as thermoplastic resins including polyetherimide (PEI), polyethylene terephthalate (PET), and polyethylene (PE), as well as polyimide resins and epoxy resins; glasses; ceramics; and papers.

### <Oxidoreductase>

The oxidoreductase may be selected depending on the type of the target substance. Examples of oxidoreductases include dehydrogenases such as alcohol dehydrogenase, glutamate dehydrogenase, cholesterol dehydrogenase, aldehyde dehydrogenase, glucose dehydrogenase (GDH), cellobiose dehydrogenase, fructose dehydrogenase, sorbitol dehydrogenase, lactate dehydrogenase, malate dehydrogenase, glycerol dehydrogenase, 17B hydroxysteroid dehydrogenase, estradiol 17B dehydrogenase, amino acid dehydrogenase, glyceraldehyde 3-phosphate dehydrogenase, 3-hydroxysteroid dehydrogenase; oxidases such as glucose oxidase (GOD), galactose oxidase, bilirubin oxidase, pyruvate oxidase, D- or L-amino acid oxidase, amine oxidase, cholesterol oxidase, choline oxidase, xanthine oxidase, sarcosine oxidase, L-lactate oxidase, ascorbate oxidase, fructosyl amino acid/peptide oxidases, glycerol-3phosphate oxidase; other oxidoreductases such as diaphorase, cytochrome oxidoreductase, catalase, peroxidase, β-hydroxybutyrate dehydrogenase, and glutathione reductase.

The oxidoreductases may be oxidoreductases which uses coenzymes such as flavin adenine dinucleotide (FAD), flavin mononucleotide (FMN), pyrroloquinoline quinone (PQQ), nicotinamide adenine dinucleotide (NAD), nicotinamide adenine dinucleotide phosphate (NADP) or copper.

Depending on the type of the enzyme, the biosensor of the present invention can be used as a glucose sensor, cholesterol sensor, ethanol sensor, sorbitol sensor, fructose sensor, cellobiose sensor, lactate sensor, uric acid sensor, ketone sensor or the like.

Among these, an oxidoreductase capable of direct electron transfer with the electrode, that is, an oxidoreductase capable of direct transfer of electrons generated by the enzymatic reaction to the electrode without requiring an oxidation-reduction substance such as an electron acceptor (which oxidoreductase is also referred to as direct electron transfer-type oxidoreductase) is used.

Examples of the oxidoreductase capable of direct electron transfer with the electrode include oxidoreductases physiologically including an oxidation-reduction molecule involved in electron transfer with the electrode. For example, an oxidoreductase containing an electron transfer subunit or an electron transfer domain as the oxidation-reduction molecule may be used. Examples of the electron transfer subunit include heme-containing subunits, and examples of the electron transfer domain include heme-containing domains. Examples of the heme-containing subunits and domains include subunits and domains containing heme C or heme b, more specifically, subunits and domains containing a cytochrome such as cytochrome C or cytochrome b.

Examples of the enzyme containing a cytochrome-containing subunit as an electron transfer subunit include glucose dehydrogenase (GDH), sorbitol dehydrogenase (sorbitol DH), D-fructose dehydrogenase (fructose DH), D-glucoside-3-dehydrogenase, cellobiose dehydrogenase, lactate dehydrogenase, and urate oxidase.

Specific examples of the glucose dehydrogenase containing a cytochrome include cytochrome glucose dehydrogenase having an FAD-containing catalytic subunit (α-subunit) and a cytochrome subunit (β-subunit) (FADGDH), wherein the FADGDH preferably further has a regulatory subunit (γ-subunit) (FADGDH γαβ). Examples of the FADGDH include FAD-dependent glucose dehydrogenase derived from Burkholderia cepacia, and mutants thereof. The nucleotide sequence encoding the gamma subunit, alpha subunit, and beta subunit of FADGDH from B. cepacia is shown in SEQ ID NO: 1. The amino acid sequences of the gamma subunit, alpha subunit, and beta subunit of FADGDH from B. cepacia are shown in SEQ ID NOs: 2, 3 and 4, respectively. Examples of the mutants of FADGDH derived from Burkholderia cepacia include FADGDH mutants such as an a-subunit mutant in which the amino acid residues at positions 472 and 475 are substituted (WO 2005/103248), an a-subunit mutant in which the amino acid residues at positions 326, 365, and 472 are substituted (QYY: JP 2012-090563 A), and an a-subunit mutant in which the amino acid residues at positions 365, 326, 472, 475, 529, and the like are substituted (WO 2006/137283).

Direct electron transfer-type oxidoreductases also include an oxidoreductase which is modified with an electron acceptor and is capable of electron transfer with the electrode. The above-exemplified oxidoreductases can be modified with an electron acceptor. The electron acceptor herein may be a compound having no catalytic action which receives an electron from an oxidoreductase to undergo reduction, followed by reoxidization at the electrode. Examples of the electron acceptor include quinone compounds (for example, 1,4-naphthoquinone, vitamin K3, 9,10-phenanthrenequinone, 1,2-naphthoquinone, p-xyloquinone, methylbenzoquinone, 2,6-dimethylbenzoquinone, sodium 1,2-naphthoquinone-4-sulfonate, 1,4-anthraquinone, tetramethylbenzoquinone, and thymoquinone), phenylenediamine compounds (for example, N,N-dimethyl-1,4-phenylenediamine and N,N,N′,N′-tetramethyl-1,4-phenylenediamine), 1-methoxy-PMS (1-methoxy-5-methylphenazinium methylsulfate), PES (phenazine ethosulfate), coenzyme Q0, AZURE A chloride, phenosafranin, 6-aminoquinoxaline, and tetrathiafulvalene.

Examples of the method for the modification of the oxidoreductase with the electron acceptor include a method in which the electron acceptor is chemically bound to the enzyme (Bioelectrochemistry, 121, June 2018, 185-190, Biosensors and Bioelectronics 2020, 151, 111974). For example, the method may be a method in which a functional group such as succinimide is introduced to the electron acceptor, and the functional group is then reacted with an amino group of the enzyme to introduce the electron acceptor to the enzyme.

Examples of the method for arranging the direct electron transfer-type oxidoreductase on the electrode surface include, but are not limited to, a method in which the oxidoreductase is chemically immobilized on the electrode, a method in which the oxidoreductase is indirectly immobilized on the electrode using a conductive polymer, cross-linking agent, or the like (for example, WO 2014/002999 or JP 2016-121989 A), and a method in which the enzyme is immobilized on the electrode through a monolayer-forming molecule. Examples of the method in which the enzyme is immobilized on the electrode through a monolayer-forming molecule include a method in which the enzyme is immobilized on the electrode through a monolayer-forming molecule (SAM) disclosed in JP 2017-211383 A.

A monolayer forming molecule is a compound capable of binding to an electrode, and capable of binding the molecular recognition element, and is a compound capable of forming a monolayer when a plurality of the molecules are unidirectionally bound on the electrode surface. By using a monolayer forming molecule, the distance between the electrode and the enzyme molecule can be controlled.

The monolayer forming molecule preferably has a first functional group having affinity for the electrode, a spacer site, and a second functional group capable of reacting with a functional group of the molecular recognition element. More preferably it has a structure where the first functional group having affinity for the electrode is bound to the first end of the spacer site, and the second functional group capable of reacting with the functional group of the molecular recognition element is bound to the second end of the spacer site. Examples of the first functional group having affinity for the electrode include, when the electrode is metallic, a thiol group, and a dithiol group, and when the electrode is carbon, pyrene, and porphyrin.

Examples of the second functional group capable of reacting with a functional group of the molecular recognition element include a succinimide group when it is reacted with an amino group of the molecular recognition element (including the terminal amino group and the side chain amino group), and an oxazoline group when it is reacted with a carboxyl group of the molecular recognition element (including a terminal carboxyl group and a side chain carboxyl group).

Examples of a monolayer forming molecule having a thiol group, or a dithiol group include compounds having the following structures.

In this regard, L is a spacer, and X is a functional group capable of reacting with a functional group of the molecular recognition element. Examples thereof include a succinimide or its ester and a thiol. Examples of the type of spacer include alkylene having 1 to 20 (e.g. 3 to 7) carbon atoms, alkenylene having 1 to 20 (e.g. 3 to 7) carbon atoms, alkynylene having 1 to 20 (e.g. 3 to 7) carbon atoms, polyethylene glycol having a polymerization degree of 2 to 50, and an oligopeptide having 1 to 20 amino acid residues, or combinations of such spacers. In the alkylene, alkenylene, or alkynylene, one or more —CH₂— may be replaced by —O—.

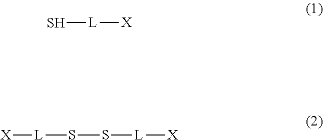

Examples of such a compound include the following DSH.

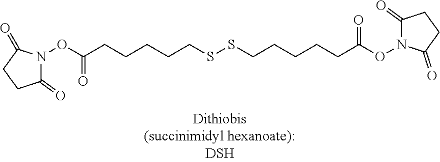 [SMILES: O=C(CCCCCSSCCCCCC(=O)ON1C(=O)CCC1=O)ON1C(=O)CCC1=O]

Examples of a monolayer forming molecule having, for example, pyrene or porphyrin include a compound having the following structure.

In this regard, Py stands for pyrene, Po for porphyrin, L for a spacer, and X for a functional group capable of reacting with the functional group of the enzyme molecule. Examples of the type of the spacer include alkylene having 1 to 20 carbon atoms, alkenylene having 1 to 20 carbon atoms, alkynylene having 1 to 20 carbon atoms, polyethylene glycol having a polymerization degree of 2 to 50, and an oligopeptide having 1 to 20 amino acid residues.

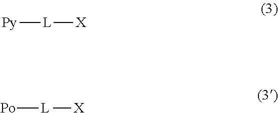

Examples of a monolayer forming molecule having, for example, pyrene include a compound having the following structure.

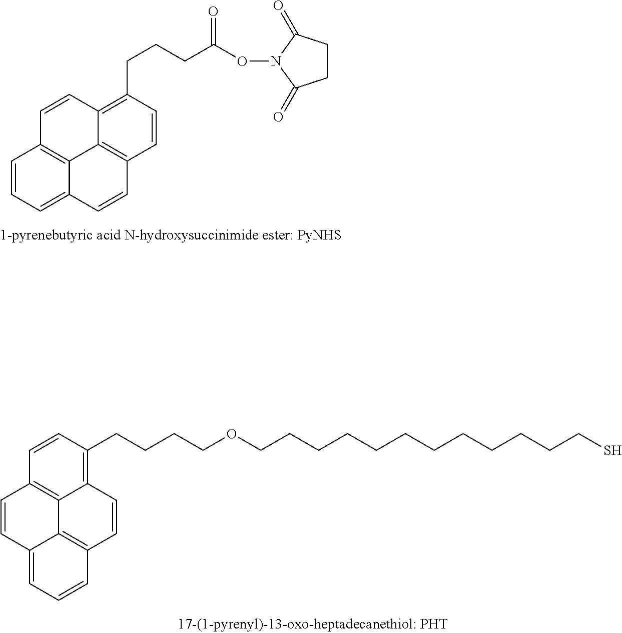 [SMILES: O=C(CCCc1ccc2ccc3cccc4ccc1c2c34)ON1C(=O)CCC1=O ; SCCCCCCCCCCCCOCCCCc1ccc2ccc3cccc4ccc1c2c34]

The enzyme electrode is prepared, for example, as follows.

First, a metal layer which functions as an electrode is formed on one side of an insulating substrate. For example, a metal layer having a desired thickness (for example, about 30 nm) is formed by depositing a metallic material, by physical vapor deposition (PVD, for example, sputtering) or chemical vapor deposition (CVD), on one side of an insulating substrate in the form of a film having a predetermined thickness (for example, about 100 μm). Instead of the metal layer, an electrode layer made of a carbon material may be formed.

To the surface of the thus obtained electrode layer, an enzyme is bound.

For example, in cases where a monolayer-forming molecule is used, the monolayer-forming molecule is first bound onto the electrode. Thereafter, by reacting a reactive functional group of the monolayer-forming molecule with an amino group or a carboxyl group of an oxidoreductase, the oxidoreductase can be immobilized on the electrode through the monolayer-forming molecule.

In cases where the enzyme is immobilized onto the electrode using a conductive polymer or a cross-linking agent, the enzyme and the reagent such as a conductive polymer or a cross-linking reagent may be added onto the electrode to prepare an enzyme electrode,

### <Reference/Counter Electrode>

The biosensor according to one embodiment of the invention also comprises a reference/counter electrode that forms a pair with the enzyme electrode. The reference/counter electrode is not limited as long as it can be generally used as a reference/counter electrode for a biosensor. Examples of the reference/counter electrode include a silver/silver chloride electrode and a calomel electrode.

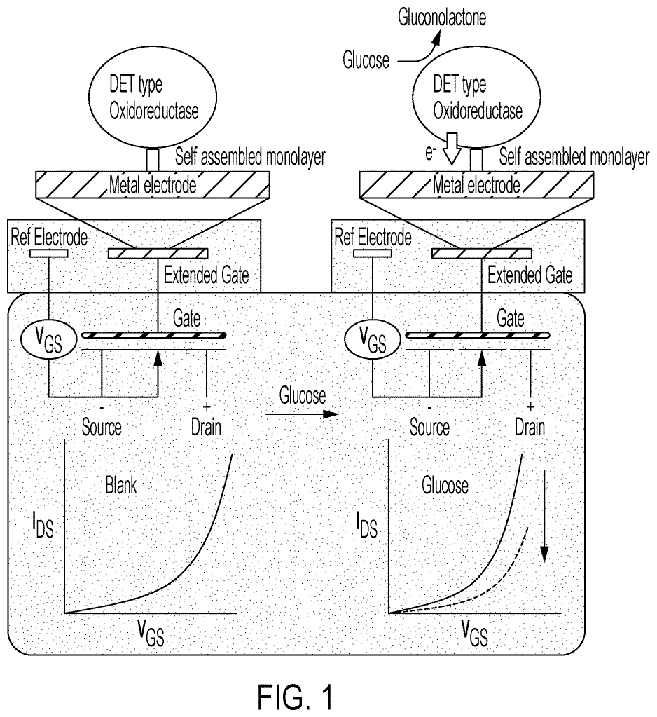

Hereinafter, one embodiment of the biosensor is explained with reference to FIG. 1.

However, the biosensor of the invention is not limited to this embodiment.

As shown in FIG. 1, the enzyme electrode is connected to the gate electrode of the FET. The FET has a drain electrode and a source electrode in addition to the gate electrode and the potential between the source and the drain (V_{DS}) is kept to a constant value.

The enzyme electrode is installed in the reaction zone together with the reference/counter electrode, and a certain potential (V_{REF}) is applied, resulting in the potential between the gate and the source (V_{GS}).

During measurement, the sample to be measured is supplied to the reaction zone, and the substance to be measured in the sample undergoes a redox reaction near the enzyme electrode, which generates an electrochemical signal that is detected by the FET, causing a current to flow between the Drain and Source (I_{DS}).

### <Method of Measuring a Target Substance>

A measuring method according to one embodiment of the invention comprises bringing a sample containing a measuring target substance into contact with the biosensor as described above and measuring a signal generated via a reaction between the substance and the oxidoreductase, such as a current flowing between the source electrode and the drain electrode to thereby calculate the concentration of the target substance based on the signal.

The step of bringing a sample containing a target substance into contact with a biosensor may be either a step of adding the sample dropwise to the biosensor or a step of immersing the biosensor in the sample. In cases of an implantable sensor, the method also includes a step of implanting the sensor in the body to place the sensor in a state where the sensor is in contact with a sample such as a blood sample, a sweat sample, an interstitial fluid sample or a saliva sample.

By bringing the biosensor into contact with the sample containing the target substance, oxidation reaction of the substance by the oxidoreductase can be allowed to occur, resulting in an increase in the reduced enzyme depending on the substrate concentration. More specifically, since the enzyme (DET-type oxidoreductase) is immobilized in the vicinity of the electrode, and the active center (more strictly, electron transfer unit) of the enzyme is adjacent to the electrode, its conversion from the oxidized form to the reduced form causes a change in the charge distribution in the electrode, leading to a change in the surface potential. Thereby, a junction voltage (V_{GS}) of the gate electrode of the FET, which is connected to the enzyme electrode, changes and current (I_{DS}) flows between the drain and the source of the FET. The value of this current can be a parameter which indicates the amount of the target substance. Furthermore, in addition to I_{DS} measurement, OCP (Open circuit potential: e.g., U.S. Pat. No. 11,608,515B) may also be measured steady state post the application of a certain amount of potential.

It is preferable that a certain amount of voltage is applied with respect to the reference electrode so as to yield the potential between gate and source (V_{GS}). The potential between the gate and the source (V_{GS}) is for example, −5000 mV or more and +5000 mV or less. V_{GS} may be varied or held constant during measurement.

In one embodiment, the measurement is conducted while applying a certain amount of a potential between the source and the drain. The potential between the source and the drain (V_{DS}) is for example, −5000 mV or more and +5000 mV or less, −1000 mV or more and +1000 mV or less, or −500 mV or more and +500 mV or less.

The method of the present invention is applicable to either a single measurement or continuous measurement. In cases of a single measurement, for example, the sample may be brought into contact with the sensor, and then the potential may be applied, followed by measurement of the I_{DS} value. In cases of continuous measurement, for example, the sample may be brought into contact with the sensor, and then the cycle of application of the potential and measurement of the current may be repeatedly carried out at desired timing.

In the step of calculating the concentration of the target substance based on the current value, for example, a calibration curve for the sensor may be prepared by preliminarily calculating the relationship between the value of the current and the substrate concentration, and then the measured value may be applied to the calibration curve to determine the substance concentration.

### EXAMPLES

### Example 1: EG-FET for Glucose Measurement

Materials: Potassium phosphate dibasic, potassium phosphate monobasic, sodium chloride, calcium, chloride, magnesium sulfate, alumina oxide, glucose, lactate, ascorbic acid, acetaminophen, Bovine serum albumin were all purchased from Sigma Aldrich (St. Louis, MO, USA). Biologic Potentiostats were employed for all electrochemical test and purchased from (Knoxville, TN), Gold disk electrode and Ag/AgCl electrodes were purchased from CH Instruments (Texas, USA). The type of EGFET used was a N-Channel Enhancement type, product ZVNL120A. (www.mouser.com/ProductDetail/Diodes-Incorporated/ZVNL120A?qs=zhx2xqPtQQyaER2ZP0wfiw % 3D %3D)

### Preparation of Enzyme:

Recombinant Burkholderia cepacia glucose dehydrogenase (BcGDHs) comprising γαβ subunits were prepared using expression vector pTrc99A containing the structural gene for the His-tagged BcGDH according to the method in previous study for heme c maturation^{31,32}. These vectors were co-transformed into Escherichia coli strain BL21(DE3) and cultivated according to our previous reports 32,33 Co-transformed E. coli were cultured in 500 mL conical flasks containing 100 mL of ZYP-5052 medium (Protein Express. Purif. 41, 207-234 (2005)) in a rotary shaker at 30° C. for 30 h. Cells were then harvested by centrifugation, and disrupted by sonication in 10 mM HEPES buffer pH 7.8 containing 10 mM Imidazole, 500 mM NaCl, and 1% Sodium cholate. The lysate was centrifuged at 16,000×g for 20 min and the supernatant was then applied to supercentrifuge at 29,000×g for 60 min. The supernatant was applied to HisTrap HP column (Cytiva) equilibrated with 10 mM HEPES pH 7.8 containing 10 mM Imidazole, 500 mM NaCl, and 1% Sodium cholate. Protein was eluted with linear Imidazole gradient in same buffer. The active fraction was pooled and dialyzed overnight at 4° C. against PBS containing 1% sodium cholate and applied to Superdex-200 increase 10/300GL (Cytiva) equilibrated with PBS containing 1% sodium cholate. Pooled fractions were dialyzed at 4° C. against 10 mM potassium phosphate buffer containing, pH 7.0. Purity of purified protein was confirmed by SDS-PAGE.

### Extended Gate Electrode Preparation:

The extended gate electrode was prepared by polishing gold disk electrode using 0.3 and 0.05 μm alumina oxide powder, followed by chemical washing using piranha acid. After the chemical wash the electrodes were electrochemically cleaned using 50 mM KOH by sweeping voltage from 0.0V to ~1.2V at 50 mV/sec until the response current was stable. The electrode was then incubated in 100 μM DSH for 18 hours while being shaken at 600 RPM at 25° C. 0.014 mg/mL BcGDH was then added onto each electrode for 24 hours while being agitated at 600 RPM and 25° C. Once complete the electrodes were stored in 100 mM potassium phosphate buffer (PPB) at pH 7.04 solution until further use.

### Electrochemical Testing Procedures:

The testing of each EGFET was performed using either 100 mM PPB at pH 7.04, artificial sweat, or artificial serum depending on the type of test. For all testing the reference electrode was a single junction Ag/AgCl, and the solution volume was 10 mL, being well mixed using a magnetic stir rod at 250 RPM. The artificial sweat consisted of the following components: 5.5 mM sodium chloride, 13.28 mM calcium, chloride, 0.24 mM magnesium sulfate, 1.36 mM potassium phosphate monobasic. Artificial interstitial fluid (ISF) was made starting with the artificial sweat, but then adding an additional, 3.5 mM potassium chloride, 123 mM sodium chloride, 0.7 mM magnesium, chloride, 1.5 mM monosodium phosphate, and 7.4 mM sucrose, with an additional 533 μM bovine serum albumin (BSA) added. These ranges of both ions and proteins are within the range of bodily concentrations^{34,35}.

The electrochemical configuration was performed as follows: the potential between source and drain (V_{DS}) were held at a constant potential (0.5 V unless stated otherwise). The voltage between the gate and source (Vas) with respect to an Ag/AgCl reference electrode was either varied or held constant for continuous detection. When swept, the range was from 0.0 V to −1.0 V as a sweep rate of 20 mV/sec. For continuous detection the voltage between the gate and source was held at constant 1 V.

### Results

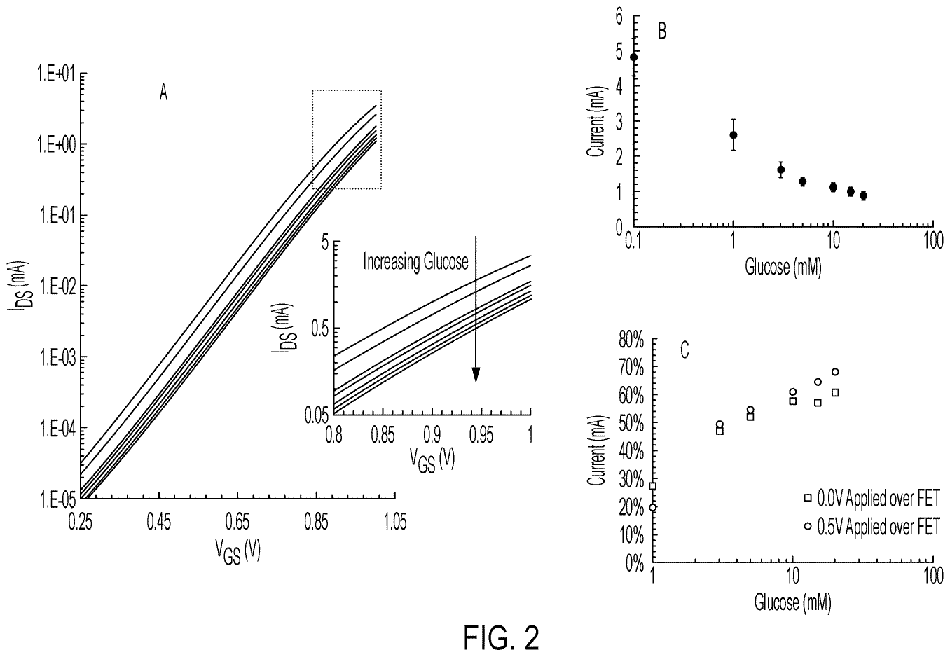

The EGFET glucose sensor was tested using a variety of solution composition, and specific interferences that may compromise the signal. First, the V_{GS} was swept from 0.0-1.0 V while measuring the I_{DS} at V_{DS} of 0.5 V to ensure the FET functioned as expected. Thereafter, glucose was added serially, ranging from 0-20 mM tested in 100 mM PPB buffer as depicted in FIG. 2, panel A. Clearly based on the figure glucose addition decreases the current flow between the source and drain electrode. Using this curve, we looked for which voltage gave the largest relative change I_{DS} to then develop our calibration curve and apply for future continuous monitoring. FIG. 2, panel B shows the calibration curve using 3 EGFET sensor while varying glucose from 0 mM to 20 mM at the designated gate voltage. The calibration curve was calculated by taking the measured current between the source and drain I_{DS} when the source and gate voltage reached 1 V. The standard deviation is derived off using 3 replicates in 100 mM PPB solution. FIG. 2, panel C shows how changing the voltage between the source and drain does not change the relative change in signal, only the absolute signal measured. Based on FIG. 2, panel C we chose to use 0.5V between the source and drain for the rest of the experimental design to ensure continuity over the test, although optimization of the applied voltage between the source and drain depended heavily on the FET chosen.

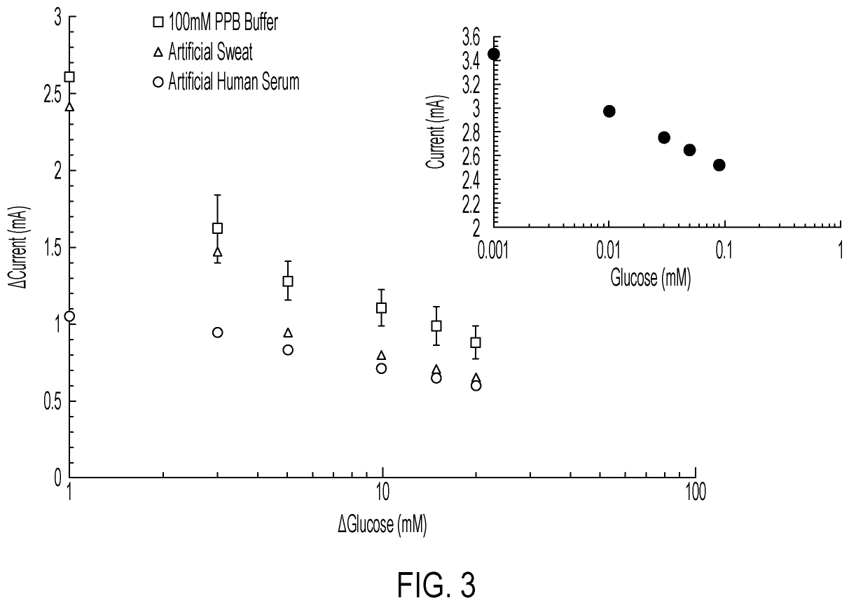

FIG. 3 shows the impact of varying solution composition on the calibration curve. The various solutions compared include 100 mM PPB, artificial sweat, and artificial human serum. The correlation was very strong for all three compositions, being 0.93, 0.96, and 0.98 for 100 mM PPB, artificial sweat, and artificial serum respectively. Although the slope did decay going from buffer to serum, being −0.84 for buffer, −0.74 for artificial sweat, and −0.15 for artificial serum. The solution composition further impacted the initial current flow between the source and drain. This may be related to the ionic strength, or net ionic charge of the solution, which can directly affect the voltage threshold over the gate electrode. The FIG. 3 inset shows testing in artificial sweat using glucose ranging from 10-90 μM which is representative of sweat concentrations.

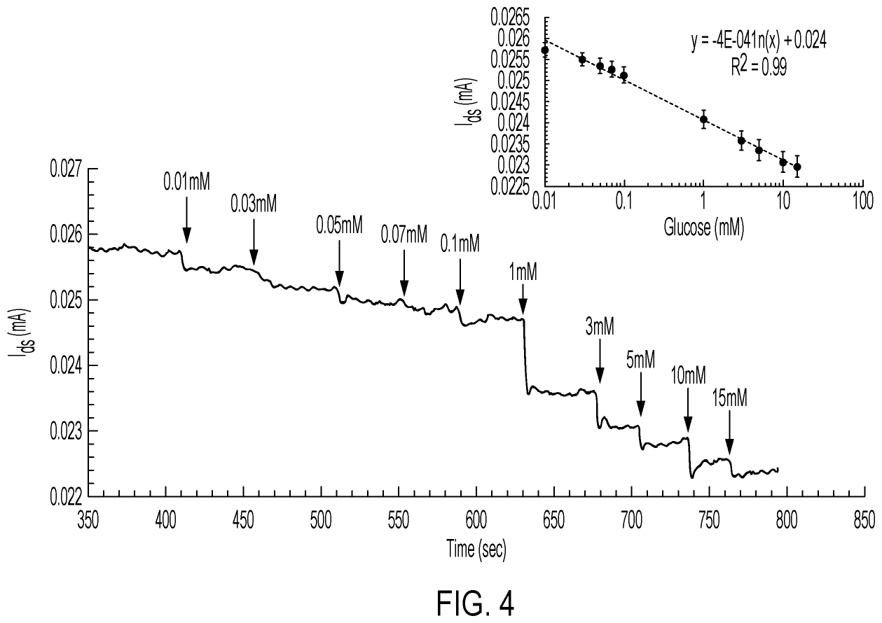

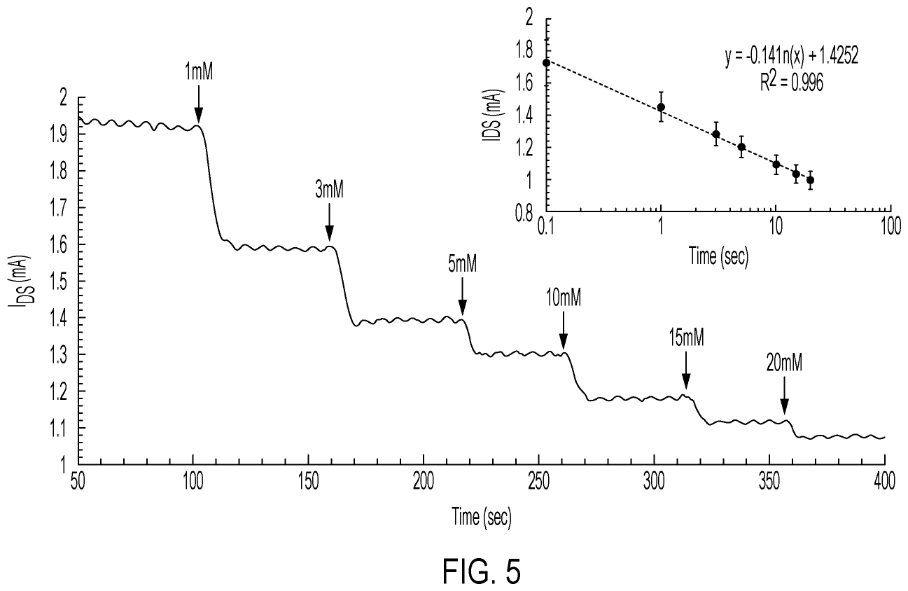

The EGFET sensor was then tested in a continuous platform using artificial sweat and ISF as the solution composition. The voltage between the gate electrode and source was held at 1 V for 10 minutes to allow for stabilization of the response current between the source and drain electrode. Once the current was stable (less than 10% change over 1 minute) glucose was serially added from 0.01 mM up to 20 mM, depending on the target complex (0.01 mM-15 mM for sweat, and 1 mM to 20 mM for artificial ISF). response was then translated into a calibration curve by taking a 1-minute average after each addition of glucose. The slope and correlation of the sweat solution was 4E-4 mA/mM glucose, and 0.99 respectively while for artificial ISF the response was much greater with a slope of 0.14 mA/mM glucose, with a 0.99 correlation. FIGS. 4 and 5 show the results for both the continuous testing, and the corresponding calibration curves using 3 replicates for each study. The difference in baseline may be attributed to changes in the EGFET transistor, as each as different baseline currents, as well as using different reference electrode which may shift the apparent voltage threshold.

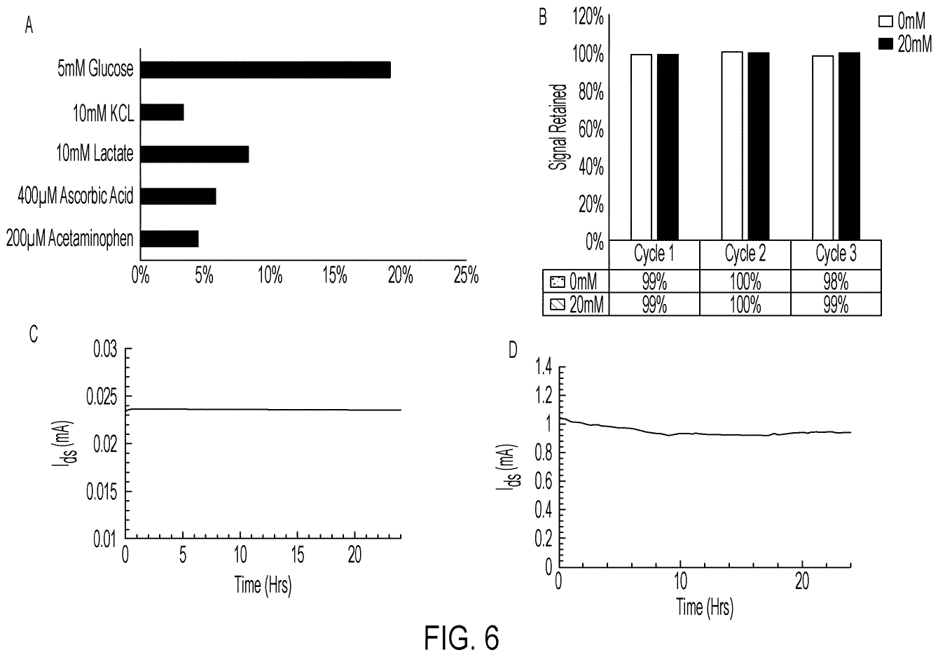

Evaluation to test various interference that may compromise the signal measured between the source and drain electrode. FIG. 6, panel A shows various endogenous, and exogenous substrates tested, including addition of 10 mM KCl, 10 mM lactate, 400 μM ascorbic acid, and 200 μM acetaminophen were tested at concentrations all above EP7-A2 Guidelines. The test was performed by holding the voltage between the gate and source at a constant 1.0 V. After 10 minutes of incubation 3 mM glucose was added and allowed to reach steady state. Each interferent was then serially added, starting from 10 mM KCl, down to 200 μM acetaminophen. After each addition the signal was allowed to incubate for 5 minutes, then a 30 second average was taken of the current between the source and drain electrode. The precent change in signal was calculated by taking a 30 second average current measured after each interference was added relative to the baseline 3 mM glucose response. Finally, the glucose was raised up to 5 mM, demonstrating the sensor is still glucose dependent, and the relative change from glucose is much greater than the interference. This test was performed using artificial ISF, with the addition of 533 μM BSA to match physiological levels. Artificial ISF with BSA was chosen for the interference study in to include impacts such as physical adsorption to the electrode surface, although in a wearable format this type of interference would be much less prominent. The specific interfering substrate where selected to test different types of interference that may occur. Both ascorbic acid, and acetaminophen can be oxidized at the electrode surface, causing noise, while KCl, and lactate may alter the reference electrode potential, or pH of the local electrode surface. Based on this all the interfering molecules caused negligible change in the signal, having less change than the background noise. To ensure that the EGFET was reversible, and could be used over time, the sensor was placed in alternating solutions of 0 mM and 20 mM, and signal retention was calculated based the initial values found in FIG. 3. The results of this shown in FIG. 6, panel B demonstrate that the sensor retained the ability to measure changes in glucose between 0 mM and 20 mM repeatedly, while retaining over 98% of the initial signal. One advantage of using the EGFET compared to conventional OCP type continuous glucose sensor is that there is no need to “reset” the enzyme through applying an oxidizing potential. This may be since there is a potential held between the source electrode and gate, which will oxidize the enzyme, through the gate leakage current of the FET. The ZVNL120A has a 20 nA gate source leakage current based on the spec sheet, which equates to about 61 μM of enzyme being oxidized over the 5-minute period. When using conventional OCP, there is no applied voltage, and most potentiometers can measure current below pA ranges which in the same 5-minute period would be sub-picomolar concentrations of enzyme.

The acute stability was looked at for both artificial ISF, and artificial sweat. This was done by using the respective medium, with 20 mM glucose and letting the sensor run continuously for 24 hours. The drift was calculated over each hour, helps give an initial understanding into the stability of the system. We did not measure long term stability, as we have already previously monitored the stability for the enzyme for 3 months in an OCP configuration³⁶. When in the artificial sweat at 20 mM glucose the drift in current/hour is 1E-6 mA/hr which equates to 0.06 mM change in glucose over the 24 hours. FIG. 6, panel C shows the stability in artificial sweat, and FIG. 6, panel D shows the stability in artificial ISF. Stability was poorer in the artificial ISF solution, losing about 11% of the signal over the first 8 hours before becoming steady state. After the first 8-hour drift, the signal was steady, decreasing by only 1% over the next 16 hours. The total drift in artificial ISF over the 24 hours was 10.7%, with a change of 0.0046 mA/hr equating to a change in 0.03 mM glucose over the 24-hour test period. Both tests demonstrate that the sensor has enough stability to maintain a reliable signal over the period tested.

### Discussion

The slopes for each of the responses were different which impacted the LOD for each composition. Based on these slopes, the limit of detection (LOD) for each of the solutions ranges being 63.3, 71.8, and 354 μM glucose for buffer, sweat and serum respectively. The LOD was calculated by measuring 5 blank electrodes in each buffer and taking 3*STD divided by the slope for the respective buffer. This shows the versatility of using the EGFET paired with DET-FADGDH which changes the electrode junction potential.

The continuous application for glucose testing is much more practical, and therefore we evaluated the ability for the EGFET to measure glucose continuously using the immobilized DET-FADGDH enzyme. As mentioned during the continuous test the V_{DS} was 0.5 V, and the V_{GS} was held constant at 1 V. When using artificial sweat as the complex medium, the sensor was allowed to equilibrate for 10 minutes, during which time the current between the drain and source decreased down to about 26 ρA. While when using artificial ISF solution the steady state 0 mM glucose I_{DS} was approximately 1.9 mA much higher than that of the artificial seat. Both current response curves were less then when using linear sweep voltammetry (FIG. 3) where the current reached nearly 2.2 mA using artificial sweat or artificial ISF at 0 mM glucose. We attributed these decreases and variation to several aspects, first to linear sweep voltammetry being a potentiodynamic process, measuring changes in both faradaic, and non-faradaic processes, where holding a constant voltage will only measure faradaic changes between the electrode³⁷. This is seen similarly when measuring cyclic voltammetry, versus amperometry which will have less current flow between the working and counter due to being merely faradic versus both faradaic and non-faradaic. Additional factor is that each FET has slight variation, and shifts in the voltage threshold, which will be reflected in the I_{DS} over the electrode. Since the impact is exponential, a small change or shift of 20-50 mV could be reflected in several hundreds of μA. The two other possibilities for variation in the measurement mediums (artificial sweat and ISF) is the ionic strength of each solution is largely different. OCP changes are highly impacted by the Debye length of the electrode, which is directly influenced by solution ionic strength. These changes will alter the initial baseline current between the source and drain electrode. The final source of variation is due to the design of the electrodes, using gold disk electrodes gives innate variability based on the user's skill, which may be reflected in the error or change in baseline. The response for both mediums (artificial sweat and ISF) was still glucose dependent, and had rapid changes upon each additional glucose concentration, from 0 mM up to 20 mM. Replicates of 3 electrodes were tested for continuous measurement using both sweat glucose concentration ranges (0.01 mM-0.1 mM) and ISF concentrations up to 20 mM.

For detection in sweat the slope was lower than that of using linear sweep voltammetry, but the LOD was 29.8 μM glucose which was better than the LOD calculated based on the linear sweep voltammetry test and is sufficient to measure changes in both sweat/serum glucose. The calculated LOD when measuring in artificial ISF was found to be 53.6 μM which is comparable to the measurements taking linear sweep voltammetry. Both these values are sufficient for the detection of glucose in either artificial sweat or artificial ISF. The LOD was calculated for the continuous sensor by measuring 1 minute of the blank electrodes (3 replicates), then taking 3*STD and dividing by the respective slope. To evaluate the reversibility of the system, we alternated the concentration from 0 mM to 20 mM and back 3 times after the continuous test. The change in signal for either 0 mm or 20 mM was less than 2% over each cycle as shown in FIG. 6B. This clearly demonstrated the ability of the sensor to be compatible with continuous monitoring as the blood fluctuations for glucose range from 2.6 mM up to 21 mM which is easily encompassed within our sensor's capability²¹. While using sweat we expect a range of glucose to be about 1% of that in plasma or ISF (ranging from 0.02-0.2 mM) which was also within the range of the demonstrated glucose sensor. Further, the sensor showed little saturation, and had immediate changes in signal response (less then 10 seconds to reach saturation for each concentration).

The total change from 0 mM to 15 mM was about 3.1 mA which based on the FET employed specification sheet reflects a change in approximately 100 mV over the V_{TH}. This change is consistent with our previous reported work when using conventional OCP to measure the change in junction potential across the electrode. This suggests that the change driving the reaction is like that of OCP, where the enzyme is reduced, but is not immediately re-oxidized with either an external voltage, or through auto-oxidation occurring with the electrode surface. This also shows why the Ios calibration curve fits a logarithmic slope, compared to a linear which most amperometric sensors are fitted with. Since OCP is a potentiometric sensor, the response is governed by the Nernst equation as shown in equation 3. This will then be reflected in the V_{Th} in a logarithmic manner, and in turn the current flow between source and drain electrode.

The last key characterization tested was that of electroactive interference, and stability. We chose to test several types of interference, substrates that may impact the electrode directly through oxidation/reduction, as well as those that may interact with the solution dipole, or reference electrode. To compare the change in signal caused be the different interfering compounds, we added an additional 2 mM glucose to increase the concentration from 3 mM to 5 mM as a signal comparison. 10 mM KCl, 10 mM lactate, 400 μM ascorbic acid, and 200 μM acetaminophen were added to represent endogenous and exogenous substrates. Based on FIG. 6A the interference all altered the signal of 3 mM no more then 9% of the initial 3 mM current response. Overall, none of the tested interference had a significant impact on the signal, which is expected considering that the signal change measured at the gate electrode is mostly driven by changing in the junction potential. Lactate, ascorbic acid, and acetaminophen will not change the overall pH, or dipole of the solution, further these substrates should not be oxidized at the electrode surface since there is no potential between the gate electrode and the drain. KCl was tested to see if these was an impact on the voltage across the reference since the electrode which may also shift the voltage threshold for the sensor. This impact was not seen and had a negligible impact on the response. After each interference was added, the glucose was increases to 5 mM, which had nearly a 2.5× increase in signal change than any of the interfering substrates. We did not directly investigate substrate specificity, as our group has previously reported and characterized this using DET-FADGDH enzymes looking at competing sugars, and other nonspecific substrates¹⁶. Although this is not expected to impact the sensor signal since the enzyme employed has very high glucose specificity.

Stability was tested by using either artificial sweat or artificial ISF and measuring the I_{DS} over 24 hours with the addition of 20 mM glucose. This acute stability is helpful to understand the drift over time, while showing that the FET maintains a constant source-drain current. The stability was excellent in artificial sweat, having a drift of only 0.1% over the 24 hours which suggest the signal fidelity would be sufficient over the time needed. As for the artificial ISF there was less stability in the first 8 hours before reaching a steady state over the next 16 hours. This initial drift may be attributed to adhesion of BSA over the electrode since we did not have any blocking agents, or membrane to protect the surface. This could be possibly mitigated by the addition of blocking agents such as ethanolamine, or preblocking in a BSA type solution. We did not evaluate that long term stability of the enzyme for two main reasons, first is the application we propose is acute continuous monitoring of glucose in a wearable form factor for sweat or ISF. Second is that we have previously demonstrated that the enzyme is stable for 3 months under OCP conditions³⁶.

This work demonstrates the first development of an EGFET using DET-FADGDH enzyme. We looked at 3 different buffer modalities, ranging from 100 mM PPB, artificial sweat, and artificial human serum. Showed the ability to measure glucose both continuously and reversibility in artificial sweat, as well as being able measure glucose down to ~50 μM concentrations in artificial sweat and ISF. Finally, the impact of key interfering ions and substrates were evaluated compared to glucose. The outcome of this was that the impact of interference was very little to negligible compared to the change in I_{DS} from glucose fluctuations.

### Example 2: EG-FET for Lactate Measurement

### Materials

Potassium phosphate dibasic, potassium phosphate monobasic, sodium chloride, calcium chloride, magnesium sulfate, alumina, D-glucose, sodium l-lactate, ascorbic acid, acetaminophen, sulfuric acid, 30K centrifuge filter tubes, and hydrogen peroxide were all purchased from Sigma Aldrich (St. Louis, MO, USA), VSP-300 Potentiostats were employed for all electrochemical measurements and purchased from Biologic (Knoxville, TN, USA). Gold disk electrodes and Ag/AgCl electrodes were purchased from CH Instruments (Austin, TX, USA). An N-channel, enhancement mode MOSFET (ZVNL120A) was used for the EGFET sensor (Diodes Incorporated, Plano, TX, USA). Amine-reactive phenazine ethosulfate was purchased from Dojindo Laboratories (Kumamoto, Japan).

### Preparation of Enzyme:

Aerococcus viridans lactate oxidase was prepared as previously described and was modified using arPES (Biosens Bioelectron. 2020 Mar. 1:151:111974)^{28,24}. Briefly, to conjugate arPES to AvLOx, 50 mM arPES was incubated with 1.6 mg/mL AvLOx and agitated at 1200 RPM for 25 minutes using a thermomixer at 25° C. Residual arPES was filtered using a 30K Millipore centrifuge filter tube at 4° C. and 16,000 G for 5 minutes.

### Extended Gate Electrode Preparation:

Extended gate electrodes were prepared by polishing gold disk electrodes using 0.3 μm and 0.05 μm alumina powder, followed by chemical washing using piranha acid (3:1 ratio of concentrated sulfuric acid to 30% hydrogen peroxide). After washing, electrodes were electrochemically cleaned in 50 mM KOH by applying a swept potential from 0.0 V to −1.2 V vs. Ag/AgCl at 50 mV/sec until stable voltammograms were obtained. Electrodes were then incubated at 25° C. in 100 μM dithiobis(succinimidyl hexanoate) (DSH) for 18 hours while being shaken at 600 RPM. 0.014 mg/mL arPES-conjugated AvLOx was then added onto each electrode for 24 hours while being agitated at 600 RPM and 25° C. Once complete the electrodes were stored at 4° C. in 100 mM potassium phosphate buffer (PPB) at pH 7.4 until further use.

### Electrochemical Testing Procedures:

Electrochemical characterization was performed using both channels of a bipotentiostat, as follows: with the FET source terminal grounded, a constant potential (0.5 V unless stated otherwise) was applied to the FET drain to provide a drain-source bias (V_{DS}). The FET gate was connected to the enzyme-functionalized working electrode to form the extended gate electrode. Simultaneously, a potential (V_{Ref}) was applied to an Ag/AgCl reference electrode which, along with the extended gate electrode and the electrolyte solution, formed the complete electrochemical cell. V_{Ref} was either varied from 0.0V to 1.5V (20 mV/s scan rate) or held constant at 1.5V for continuous detection. The drain-source bias, coupled with the gate-source potential (V_{GS}) resulting from V_{Ref} and the current state of the electrochemical cell, influence the measured I_{D}. Electrodes were tested using either an artificial sweat solution (2.5 mM calcium chloride, 3.5 mM potassium chloride, 123 mM sodium chloride, 0.7 mM magnesium chloride, and 1.5 mM monosodium phosphate) or 100 mM PPB^{34,35}.

### Results

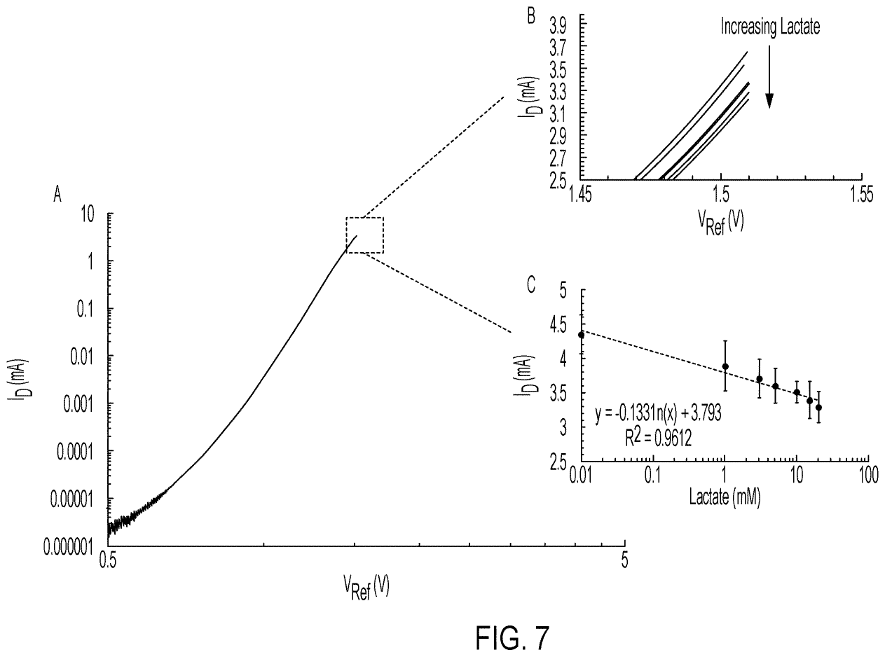

The EGFET sensor was first evaluated by sweeping V_{Ref} between 0.0 V and 1.5 V while applying a constant 0.5 V drain-source bias. Between sweeps, lactate was added to the PPB-containing electrochemical cell to generate final concentrations between 0 mM-20 mM. FIG. 7, panel A shows the measured I_{D} while sweeping V_{Ref} from 0 V-1.5 V. FIG. 7, panel B shows a magnified view of the upper range of the same plot contained within the indicated region. As lactate is added into the solution, the FET V_{GS} decreases, lowering the FET channel conductivity and limiting the measured drain current at a given drain-source bias potential. FIG. 7, panel C shows a calibration curve for lactate measured in artificial sweat ranging from 0 mM-20 mM, recording I_{D} at a fixed V_{Ref} (1.5V). These results clearly show that I_{D} (and V_{GS}) shift in a lactate-dependent manner, with a sensitivity of −0.133 mA/(decade mM lactate). The LOD (2.12 mM) was calculated by taking 3 times the standard deviation of the measured signal and dividing this figure by the response slope over the measured lactate range.

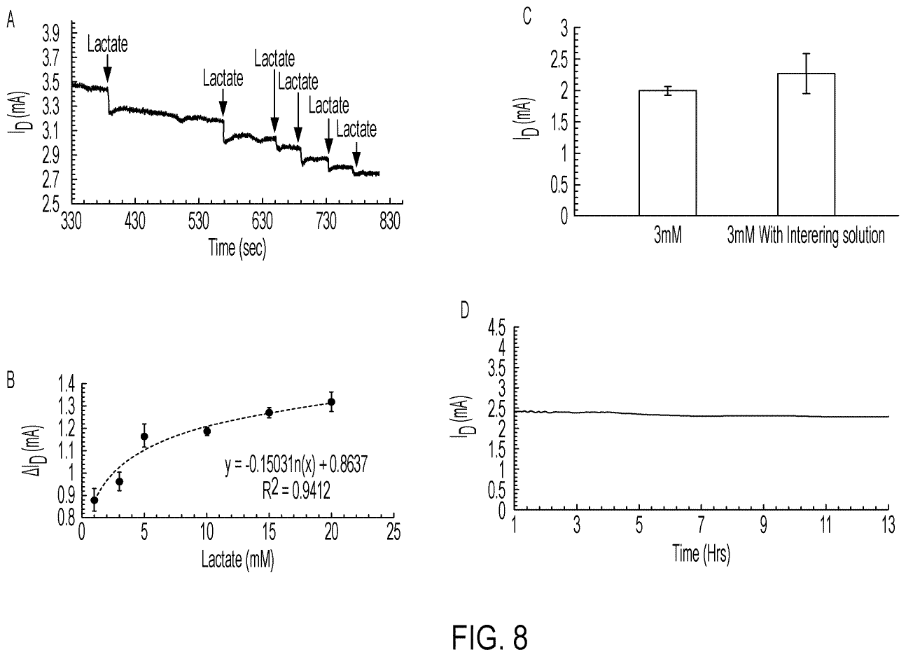

To further characterize the EGFET sensor using arPES-modified AvLOx, we measured the sensor response to continuous additions of lactate, evaluated the effect of serval interferent species on lactate detection, and investigated the stability of the sensor over 12 hours while maintaining a constant lactate concentration. FIG. 8, panel A shows an example of continuous lactate sensing as lactate was incrementally added to the solution over the course of several minutes. In this experiment, the sensor was immersed in artificial sweat, and the solution lactate concentration was sequentially increased from 0 mM-20 mM while holding V_{Ref} at 1.5 V and V_{DS} at 0.5 V. This response was then used to generate a calibration curve as seen in FIG. 8, panel B where the measured change in drain current (ΔI_{D}) was obtained for each of the lactate concentrations tested. The calculated LOD of this sensor was found to be 1.01 mM, with a correlation of 0.96 over the measured range. The LOD was calculated by taking 3 times the standard deviation of the blank electrode, divided by the slope measured over the entire range of lactate concentrations tested. FIG. 8, panel C shows the impact of the presence of several interferent species on lactate detection. For this experiment, the response of the sensor was compared between two solutions containing 3 mM lactate in artificial sweat, with and without a mixture of interferent species (400 μM ascorbic acid, 200 μM acetaminophen, 10 mM glucose, and 10 mM potassium chloride) respectively³⁸. These interferents were selected to evaluate the impact of surface reactions which can occur through direct electrooxidation of ascorbic acid and acetaminophen. Additionally, the addition of 10 mM potassium chloride was intended to perturb the reference electrode and/or change the ionic strength of the solution, thereby influencing the Debye length at the electrode surface and modifying the extended gate electrode potential³⁷. This experiment was performed with V_{Ref} held at 1.5 V, and V_{DS} held at 0.5 V. After the addition of 3 mM lactate and measurement of the steady-state drain current, the interferent mixture (10 μL total to minimize dilution of lactate) was added to the solution. The system was allowed to run while stirring for 5 minutes, and the steady-state drain current was again measured. This was validated using three electrodes each for the artificial sweat solution with and without added interferent species, respectively. There was no statistically-significant difference in the signal measured for 3 mM lactate when compared to the addition of interfering specific (p-value: 0.51) suggesting that the sensor maintained a lactate-specific response. For the final characterization experiment, sensor drift was measured over a 12-hour use period. To evaluate stability, the immobilized electrode was soaked in 20 mM lactate for 12 hours and I_{D} was continuously monitored holding V_{Ref} at 1.5 V and V_{DS} at 0.5 V. The overall drift was approximately 0.0071 mA/hour over the 12-hour period, indicating a ~5% change in measured ID.

An EGFET sensor based on the reaction of lactate and arPES-modified AvLOx has yet to be reported or characterized as a platform for wearable sensing. Here we present the characterization of an EGFET sensor for measuring lactate in an artificial sweat medium. FIG. 7, panel A shows the entire I_{D} vs V_{Ref} curve with V_{Ref} swept between 0 V-1.5 V with varying concentrations of lactate. These results clearly show that the measured drain current is lactate-dependent, decreasing with increasing lactate concentrations. This change is due in part to the interaction of arPES-modified AvLOx with lactate, as well as local changes to the solution surrounding the electrode caused by production of pyruvate. Upon reacting with lactate, the arPES mediator is reduced, changing the ratio of oxidized arPES to reduced arPES on the extended gate electrode and modifying the effective threshold voltage of the EGFET.

### Continuous Detection of Lactate:

To test the ability of the arPES-modified AvLOx sensor to measure lactate continuously, we sequentially added 0 mM-20 mM lactate into an artificial sweat solution while applying a fixed V_{Ref} and V_{DS}. For V_{Ref}, 1.5 V was selected based on the relatively large drain current observed at this potential. Based on the results shown in FIG. 7, panel C, the observed sensitivity to lactate was −0.133 mA/(decade mM lactate) with a LOD of 2.12 mM lactate. One challenge arising from the use of semiconductor FETs stems from innate inter-transistor variability in Vₜₕ; this results in low background reproducibility (signal obtained in the absence of lactate), leading to a poor LOD. To improve the sensor response, reproducibility, and performance, we chose to normalize calibration results by performing background subtraction using the blank signal obtained with each electrode (in the absence of lactate), thereby mitigating some degree of background noise and variability. Based on the calibration curve shown in FIG. 8, panel B (using ΔI_{Dd} versus Δ[lactate]) the LOD drops down to 0.98 mM upon normalizing to the blank. This is due to most of the observed signal variability being accounted for within the 0 mM blank measurement, while the relative change for each lactate addition was similar. To calculate the LOD of the ΔI_{D} vs Δ[lactate] measurements, we used the same equation of dividing the slope by the standard deviation of the 1 mM lactate signal multiplied by 3.

### Interference Impact:

A key attribute of electrochemical sensors is the impact of the presence of interferent species on the measurement of the analyte of interest at the working electrode. Traditional amperometric sensors suffer heavily from the presence of electroactive endogenous and/or exogenous species since these interferents are oxidized or reduced at smaller potentials than the overpotential required to sense the analyte of interest; as a result, these contribute to the recorded current along with the desired analyte. Most lactate sensors that rely on lactate oxidase (Lox) require an overpotential of 0.4 V or greater when a gold or platinum electrode is used, to oxidize hydrogen peroxide (the product of the enzymatic conversion of lactate by Lox). This high overpotential leads to the spontaneous oxidation of other species such as ascorbic acid or acetaminophen which may be present in the sample (particularly in the case of biological fluids). Since EGFET sensors are based on the Nernst equation (as is the case with “traditional” OCP sensors) no overpotential is applied to the working electrode, greatly mitigating the impact of these electroactive interferents. On the other hand, potentiometric sensors tend to be vulnerable to changes in pH or ionic strength, which can impact the reference electrode potential or Debye length of the working electrode, thereby limiting the sensitivity of the sensor²². This effect was evaluated by mixing potassium chloride to change the ionic strength of the solution, as well as to generate direct interactions with the Ag/AgCl reference electrode. The final interferent tested was glucose, which acts as a nonspecific substrate for AvLOx and may additionally bind in a nonspecific manner to the electrode surface³⁹. To test the aggregate impact of these interferent species, the sensor was evaluated in lactate-containing artificial sweat solution with and without a mixture of 400 μM ascorbic acid, 200 μM acetaminophen, 10 mM glucose, and 10 mM potassium chloride. Resulting error in the measured signal was observed as a ~12% increase in I_{D} compared to measurements performed in a solution without interferent species.

### Stability of the EGFET Sensor:

To quantify the stability of the EGFET sensor, 20 mM lactate was added into the artificial sweat solution and data was collected over a 12 hour period. Overall, the sensor showed good stability, drifting less than 0.5% ID per hour. The total drift was found to be 0.0071 mA/hr over the entire 12 hour period, representing a ~5% change in the measured drain current over the course of the experiment. This drift may be attributed to the stability of arPES which can still be auto-oxidized via reduction of oxygen, even though there is no apparent overpotential applied to the working electrode. This auto-oxidation pathway may slowly degrade some of the arPES over the 12 hour testing period. In addition, there may be limited stability or reorientation of the self-assembled monolayer employed for immobilization of the arPES-modified enzyme. These dynamic changes may impact the equilibrium of the oxidized versus reduced arPES-modified AvLOx.

This work demonstrates the first attempt at characterizing a lactate-sensitive EGFET sensor integrated with arPES-modified AvLOx which is capable of quasi-direct electron transfer²⁸. We investigated the sensor response in buffer and artificial sweat over a physiological range of lactate concentrations. Additionally, we measured the impact of several common interferent species on the measured sensor output, and determined the impact of these interferents to be negligible compared to changes in drain current resulting from changes in lactate concentration. Further, the EGFET sensor system was modeled as an equivalent circuit to better elucidate how variations in the effective threshold voltage and FET gate-source voltage resulting from changes in the working electrode junction potential impact the measured FET drain current. These results indicate that an EGFET sensing architecture may be suitable for the continuous monitoring of lactate in sweat and other bodily fluids. In addition, due to the affordable and well-characterized nature of commercially-available FETs, as well as the case of the integration of FETs into more complex electronic systems, an EGFET-based biosensing approach lends itself well to the development of multi-channel, multiplexed systems capable of measuring an array of biomarkers simultaneously.

### Example 3

### Microwire EGFET and OCP Sensor Characterization and Testing in Human Plasma

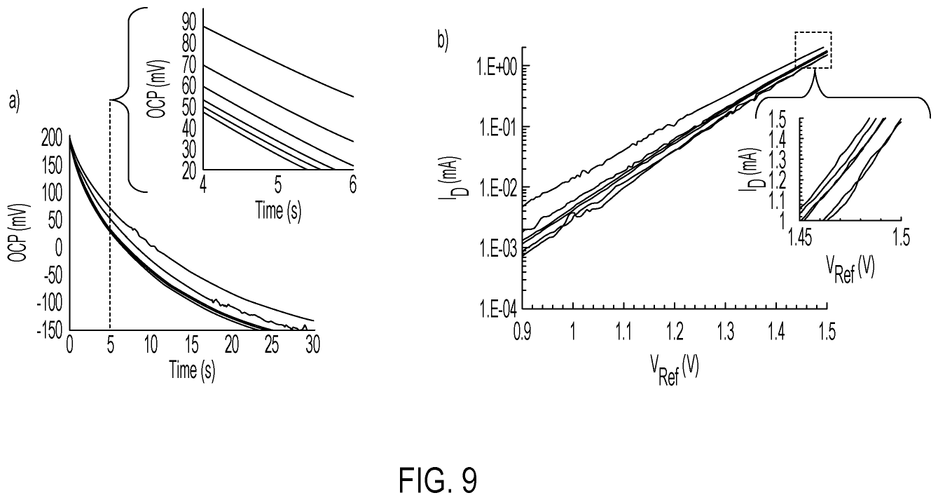

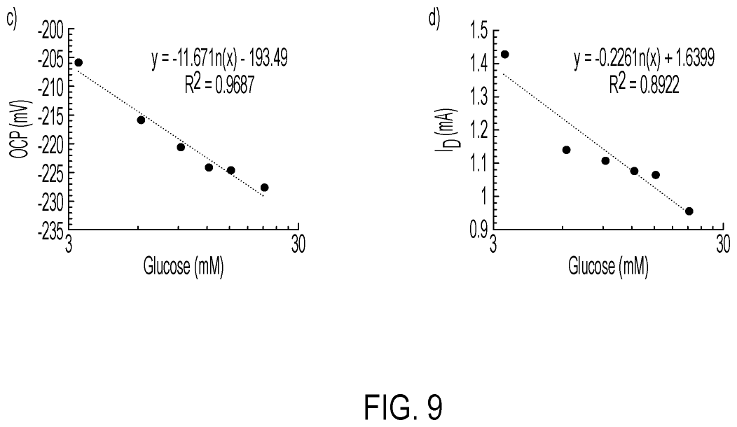

Due to the size of gold disk electrodes, they are impractical for in vivo or ex vivo application, a 2-electrode configuration using a microwire-type sensor was developed for the EGFET system. Initial functionality was demonstrated in 100 mM potassium phosphate buffer using a single junction reference electrode measuring glucose using both EFET and OCP as in Example 1. Subsequent tests involved substitution of a silver wire deposited with AgCl, in place of the original single junction reference. The sensor was tested in both EGFET and OCP configurations. FIG. 9, panels a and b show the performance of the microwire type sensor in human plasma measuring either a) OCP, or b) I_{D}. V_{ref} was swept from −1.5 V to −0.5 V across the Ag/AgCl reference electrode and source at a scan rate of 25 mV/sec using cyclic voltammetry, while a V_{DS} of −0.5 V was held between the source and drain. OCP was measured steady state post the application of chronoamperometry for 30 seconds of 0.2 V. The slope of the OCP was found to be −0.049 mV/decade mM glucose; by comparison, the slope of the EGFET response was found to be −0.049 mA/decade mM glucose. Both the OCP and EGFET measurements showed a strong correlation between the recorded sensor response and glucose concentration.

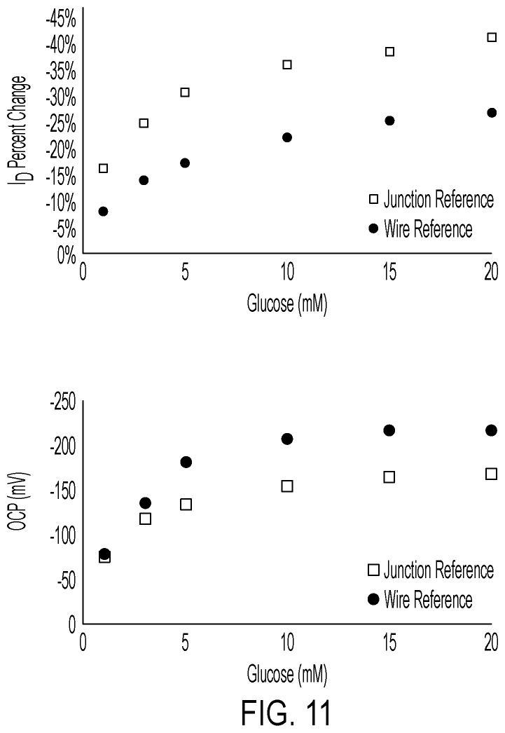

The impact of using a pseudo-reference electrode (silver wire) versus a single junction reference electrode was compared; these results are shown in FIG. 11. Using a pseudo-reference lowered the relative change in I_{D} across the range of glucose concentrations tested. This effect may be due to a shift in the cell potential (relative to the applied V_{Ref}) caused by drift in Ag/AgCl junction potential, which is less stable than the junction potential of a single junction reference electrode.

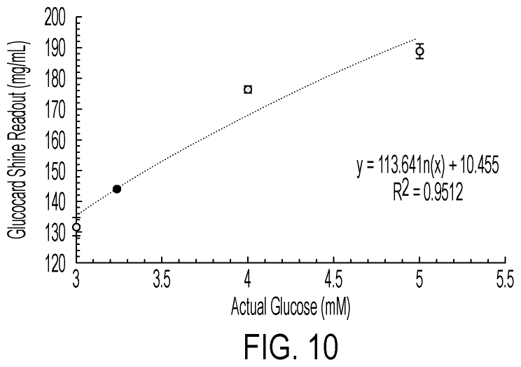

Single-donor human plasma derived from blood was used to validate the sensor in complex solutions. The initial glucose present in the plasma was quantified using a standard point of care glucose sensor (Glucocard Shine) by matching the measured mg/dL glucose to a calibration curve developed with 100 mM potassium phosphate buffer (FIG. 10). I_{D} was tested by using cyclic voltammetry to sweep V_{Ref} from −1.5-−0.5 across the Ag/AgCl reference electrode and source at a scan rate of 25 mV/sec, while V_{DS} of −0.5 V was held between the source and drain. Glucose was added serially, ranging from 3.2 mM to 23.2 mM, and once again both OCP and EGFET sensing modes were tested. Calibration curves for both OCP and EGFET experiments were obtained and showed a clear correlation between plasma glucose concentration and both OCP and I_{D} in human plasma (FIG. 9, panels c and d).

The contents of all publications including patents and patent applications as well as non-patent documents cited in the present description are hereby incorporated by reference to the same extent as if all contents were clearly described.

### REFERENCES

(1) Ferri, S.; Kojima, K.; Sode, K. Review of Glucose Oxidases and Glucose Dehydrogenases: A Bird's Eye View of Glucose Sensing Enzymes. J Diabetes Sci Technol 2011, 5 (5), 1068-1076. doi.org/10.1177/193229681100500507.
(2) Lee, I.; Probst, D.; Klonoff, D.; Sode, K. Continuous Glucose Monitoring Systems-Current Status and Future Perspectives of the Flagship Technologies in Biosensor Research-Biosensors and Bioelectronics 2021, 113054. doi.org/10.1016/j.bios.2021.113054.
(3) Quinto, M.; Losito, I.; Palmisano, F.; Zambonin, C. G. Disposable Interference-Free Glucose Biosensor Based on an Electropolymerised Poly(Pyrrole) Permselective Film. Analytica Chimica Acta 2000, 420 (1), 9-17. doi.org/10.1016/S0003-2670 (00) 00998-3.
(4) Heller, A.; Feldman, B. Electrochemical Glucose Sensors and Their Applications in Diabetes Management. Chem. Rev. 2008, 108 (7), 2482-2505. doi.org/10.1021/cr068069y.
(5) Deng, H.; Teo, A. K. L.; Gao, Z. An Interference-Free Glucose Biosensor Based on a Novel Low Potential Redox Polymer Mediator. Sensors and Actuators B: Chemical 2014, 191, 522-528. doi.org/10.1016/j.snb.2013.10.059.
(6) Okuda-Shimazaki, J.; Yoshida, H.; Sode, K. FAD Dependent Glucose Dehydrogenases-Discovery and Engineering of Representative Glucose Sensing Enzymes-. Bioelectrochemistry 2020, 132, 107414. doi.org/10.1016/j.bioelechem.2019.107414.
(7) Lee, I.; Loew, N.; Tsugawa, W.; Ikebukuro, K.; Sode, K. Development of a Third-Generation Glucose Sensor Based on the Open Circuit Potential for Continuous Glucose Monitoring. Biosensors and Bioelectronics 2018, 124-125. doi.org/10.1016/j.bios.2018.09.099.
(8) Smith, L. A.; Glasscott, M. W.; Vannoy, K. J.; Dick, J. E. Enzyme Kinetics via Open Circuit Potentiometry. Anal. Chem. 2020, 92 (2), 2266-2273. doi.org/10.1021/acs.analchem.9b04972.
(9) Song, Y.; Su, D.; Shen, Y.; Liu, H.; Wang, L. Design and Preparation of Open Circuit Potential Biosensor for in Vitro and in Vivo Glucose Monitoring. Anal Bioanal Chem 2017, 409 (1), 161-168. doi.org/10.1007/s00216-016-9982-1.
(10) You, X.; Pak, J. J. Graphene-Based Field Effect Transistor Enzymatic Glucose Biosensor Using Silk Protein for Enzyme Immobilization and Device Substrate. Sensors and Actuators B: Chemical 2014, 202, 1357-1365. doi.org/10.1016/j.snb.2014.04.079.
(11) Ahmad, R.; Ahn, M.-S.; Hahn, Y.-B. Fabrication of a Non-Enzymatic Glucose Sensor Field-Effect Transistor Based on Vertically-Oriented ZnO Nanorods Modified with Fe₂O₃. Electrochemistry Communications 2017, 77, 107-111. doi.org/10.1016/j.elecom.2017.03.006.
(12) Sarcina, L.; Macchia, E.; Tricase, A.; Scandurra, C.; Imbriano, A.; Torricelli, F.; Cioffi, N.; Torsi, L.; Bollella, P. Enzyme Based Field Effect Transistor: State-of-the-art and Future Perspectives. Electrochemical Science Adv 2022. doi.org/10.1002/elsa.202100216.
(13) Shin, D. H.; Kim, W.; Jun, J.; Lee, J. S.; Kim, J. H.; Jang, J. Highly Selective FET-Type Glucose Sensor Based on Shape-Controlled Palladium Nanoflower-Decorated Graphene. Sensors and Actuators B: Chemical 2018, 264, 216-223. doi.org/10.1016/j.snb.2018.02.139.
(14) Jung, D.-U.-J., Ahmad, R.; Hahn, Y.-B. Nonenzymatic Flexible Field-Effect Transistor Based Glucose Sensor Fabricated Using NiO Quantum Dots Modified ZnO Nanorods. Journal of Colloid and Interface Science 2018, 512, 21-28. doi.org/10.1016/j.jcis.2017.10.037.
(15) Mishra, A. K.; Jarwal, D. K.; Mukherjee, B.; Kumar, A.; Ratan, S.; Jit, S. CuO Nanowire-Based Extended-Gate Field-Effect-Transistor (FET) for PH Sensing and Enzyme-Free/Receptor-Free Glucose Sensing Applications. IEEE Sensors J. 2020, 20 (9), 5039-5047. doi.org/10.1109/JSEN.2020.2966585.
(16) Yamashita, Y.; Ferri, S.; Huynh, M. L.; Shimizu, H.; Yamaoka, H.; Sode, K. Direct Electron Transfer Type Disposable Sensor Strip for Glucose Sensing Employing an Engineered FAD Glucose Dehydrogenase. Enzyme and Microbial Technology 2013, 52 (2), 123-128. doi.org/10.1016/j.enzmictec.2012.11.002.
(17) Morrow, B.; Malkoc, A., Gong, T.; Probst, D.; Lin, C.; Sen, A.; La Belle, J. T. Development of Electrochemical Methods to Enzymatically Detect Lactate and Glucose Using Imaginary Impedance for Enhanced Management of Glycemic Compromised Patients. Critical Reviews in Biomedical Engineering 2019, 47 (3), 179-191. doi.org/10.1615/CritRevBiomedEng.2019026533.
(18) Basu, A.; Slama, M. Q.; Nicholson, W. T.; Langman, L.; Peyser, T.; Carter, R.; Basu, R. Continuous Glucose Monitor Interference With Commonly Prescribed Medications: A Pilot Study. J Diabetes Sci Technol 2017, 11 (5), 936-941. doi.org/10.1177/1932296817697329
(19) Deng, H.; Teo, A. K. L.; Gao, Z. An Interference-Free Glucose Biosensor Based on a Novel Low Potential Redox Polymer Mediator. Sensors and Actuators B: Chemical 2014, 191, 522-528. doi.org/10.1016/j.snb.2013.10.059.
(20) Ferri, S.; Kojima, K.; Sode, K. Review of Glucose Oxidases and Glucose Dehydrogenases: A Bird's Eye View of Glucose Sensing Enzymes. J Diabetes Sci Technol 2011, 5 (5), 1068-1076. doi.org/10.1177/193229681100500507.
21) Heinemann, L. Quality of Glucose Measurement with Blood Glucose Meters at the Point-of-Care: Relevance of Interfering Factors. Diabetes Technology & Therapeutics 2010, 12 (11), 847-857. doi.org/10.1089/dia.2010.0076.
22) Kesler, V.; Murmann, B.; Soh, H. T. Going beyond the Debye Length: Overcoming Charge Screening Limitations in Next-Generation Bioelectronic Sensors. ACS Nano 2020, 14 (12), 16194-16201. doi.org/10.1021/acsnano.0c08622.
(23) Vaddiraju, S.; Tomazos, I.; Burgess, D. J.; Jain, F. C.; Papadimitrakopoulos, F. Emerging Synergy between Nanotechnology and Implantable Biosensors: A Review. Biosensors and Bioelectronics 2010, 25 (7), 1553-1565. doi.org/10.1016/j.bios.2009.12.001.
(24) Hatada, M.; Loew, N.; Inose-Takahashi, Y.; Okuda-Shimazaki, J.; Tsugawa, W.; Mulchandani, A.; Sode, K., Development of a Glucose Sensor Employing Quick and Easy Modification Method with Mediator for Altering Electron Acceptor Preference. Bioelectrochemistry 2018, 121, 185-190. doi.org/10.1016/j.bioelechem.2018.02.001.
(25) Ito, K.; Okuda-Shimazaki, J.; Mori, K.; Kojima, K.; Tsugawa, W.; Ikebukuro, K.; Lin, C.-E.; La Belle, J.; Yoshida, H.; Sode, K. Designer Fungus FAD Glucose Dehydrogenase Capable of Direct Electron Transfer, Biosensors and Bioelectronics 2019, 123, 114-123. doi.org/10.1016/j.bios.2018.07.027.
(26) Sode, K.; Loew, N.; Ohnishi, Y.; Tsuruta, H.; Mori, K.; Kojima, K.; Tsugawa, W.; LaBelle, J. T.; Klonoff, D. C. Novel Fungal FAD Glucose Dehydrogenase Derived from Aspergillus Niger for Glucose Enzyme Sensor Strips. Biosensors and Bioelectronics 2017, 87, 305-311. doi.org/10.1016/j.bios.2016.08.053.
(27) Yamashita, Y.; Ferri, S.; Huynh, M. L.; Shimizu, H.; Yamaoka, H.; Sode, K. Direct Electron Transfer Type Disposable Sensor Strip for Glucose Sensing Employing an Engineered FAD Glucose Dehydrogenase. Enzyme and Microbial Technology 2013, 52 (2), 123-128. doi.org/10.1016/j.enzmictec.2012.11.002.
(28) Hiraka, K.; Kojima, K.; Tsugawa, W.; Asano, R.; Ikebukuro, K.; Sode, K. Rational Engineering of Aerococcus Viridans L-Lactate Oxidase for the Mediator Modification to Achieve Quasi-Direct Electron Transfer Type Lactate Sensor. Biosensors and Bioelectronics 2020, 151, 111974. doi.org/10.1016/j.bios.2019.111974.
(29) Hiraka, K.; Kojima, K.; Lin, C.-E.; Tsugawa, W.; Asano, R.; La Belle, J. T.; Sode, K. Minimizing the Effects of Oxygen Interference on 1-Lactate Sensors by a Single Amino Acid Mutation in Aerococcus Viridans 1-Lactate Oxidase. Biosensors and Bioelectronics 2018, 103, 163-170. doi.org/10.1016/j.bios.2017.12.018.
(30) Probst, D.; Lee, I.; Sode, K. The Development of Micro-Sized Enzyme Sensor Based on Direct Electron Transfer Type Open Circuit Potential Sensing Principle. Electrochimica Acta 2022, 426, 140798. doi.org/10.1016/j.electacta.2022.140798.
(31) Tsuya, T.; Ferri, S.; Fujikawa, M.; Yamaoka, H.; Sode, K. Cloning and Functional Expression of Glucose Dehydrogenase Complex of Burkholderia Cepacia in Escherichia Coli. Journal of Biotechnology 2006, 123 (2), 127-136. doi.org/10.1016/j.jbiotec.2005.10.017.
(32) Yamashita, Y.; Suzuki, N.; Hirose, N.; Kojima, K., Tsugawa, W.; Sode, K. Mutagenesis Study of the Cytochrome c Subunit Responsible for the Direct Electron Transfer-Type Catalytic Activity of FAD-Dependent Glucose Dehydrogenase. IJMS 2018, 19 (4), 931. doi.org/10.3390/ijms19040931.
(33) Yoshida, H.; Kojima, K.; Shiota, M.; Yoshimatsu, K.; Yamazaki, T.; Ferri, S.; Tsugawa, W.; Kamitori, S.; Sode, K. X-Ray Structure of the Direct Electron Transfer-Type FAD Glucose Dehydrogenase Catalytic Subunit Complexed with a Hitchhiker Protein. Acta Crystallogr D Struct Biol 2019, 75 (9), 841-851. doi.org/10.1107/S2059798319010878.
(34) Heikenfeld, J.; Jajack, A.; Feldman, B.; Granger, S. W.; Gaitonde, S.; Begtrup, G.; Katchman, B. A. Accessing Analytes in Biofluids for Peripheral Biochemical Monitoring. Nat Biotechnol 2019, 37 (4), 407-419. doi.org/10.1038/s41587-019-0040-3,
(35) Fogh-Andersen, N.; Altura, B. M.; Altura, B. T.; Siggaard-Andersen, O. Composition of Interstitial Fluid. Clinical Chemistry 1995, 41 (10), 1522-1525. doi.org/10.1093/clinchem/41.10.1522
(36) Lee, I.; Wakako, T.; Ikebukuro, K.; Sode, K. In Vitro Continuous 3 Months Operation of Direct Electron Transfer Type Open Circuit Potential Based Glucose Sensor: Heralding the Next CGM Sensor~. J Diabetes Sci Technol 2022, 19322968221092450. doi.org/10.1177/19322968221092449.
(37) Bard, A. J.; Faulkner, L. R. Electrochemical Methods: Fundamentals and Applications, 2nd ed.; Wiley: New York, 2001.
(38) Lorenz, C.; Sandoval, W.; Mortellaro, M. Interference Assessment of Various Endogenous and Exogenous Substances on the Performance of the Eversense Long-Term Implantable Continuous Glucose Monitoring System. Diabetes Technology & Therapeutics 2018, 20 (5), 344-352. doi.org/10.1089/dia.2018.0028.
(39) Hiraka, K.; Tsugawa, W.; Asano, R.; Yokus, M. A.; Ikebukuro, K.; Daniele, M. A.; Sode, K. Rational Design of Direct Electron Transfer Type L-Lactate Dehydrogenase for the Development of Multiplexed Biosensor. Biosensors and Bioelectronics 2021, 176, 112933. doi.org/10.1016/j.bios.2020.112933.

What is claimed is:

1. A biosensor comprising an enzyme electrode comprising a direct electron transfer type oxidoreductase wherein said enzyme electrode is electrically connected to a gate electrode of a field effect transistor (FET).

2. The biosensor according to claim 1, wherein said direct electron transfer type oxidoreductase comprises an electron transfer subunit and/or electron transfer domain.

3. The biosensor according to claim 1, wherein said direct electron transfer type oxidoreductase comprises a cytochrome containing subunit.

4. The biosensor according to claim 1, wherein said direct electron transfer type oxidoreductase is selected from the group consisting of glucose dehydrogenase, lactate dehydrogenase, sorbitol dehydrogenase, D-fructose dehydrogenase, D-glucoside-3-dehydrogenase, and cellobiose dehydrogenase.

5. The biosensor according to claim 1, wherein said direct electron transfer type oxidoreductase is an oxidoreductase which is modified with an electron acceptor.

6. The biosensor according to claim 5, wherein said electron acceptor is selected from the group consisting of 1,4-naphthoquinone, vitamin K3, 9,10-phenanthrenequinone, 1,2-naphthoquinone, p-xyloquinone, methylbenzoquinone, 2,6-dimethylbenzoquinone, sodium 1,2-naphthoquinone-4-sulfonate, 1,4-anthraquinone, tetramethylbenzoquinone, and thymoquinone), phenylenediamine compounds (for example, N,N-dimethyl-1,4-phenylenediamine and N,N,N′,N′-tetramethyl-1,4-phenylenediamine), 1-methoxy-PMS (1-methoxy-5-methylphenazinium methylsulfate), PES (phenazine ethosulfate), coenzyme Q0, AZURE A chloride, phenosafranin, 6-aminoquinoxaline, and tetrathiafulvalene

7. The biosensor according to claim 1, wherein said biosensor is a glucose sensor or a lactate sensor.

8. The biosensor according to claim 1, wherein said biosensor further comprising a reference electrode.

9. A measuring method comprising:
bringing a sample containing a measuring target substance into contact with the biosensor as described above, and
measuring a signal generated via a reaction between the substance and the oxidoreductase.

10. The method according to claim 9, wherein the measuring target substance is glucose or lactate.

11. The method according to claim 9, wherein the sample is selected from a blood sample, a sweat sample, an interstitial fluid sample or a saliva sample.

12. The method according to claim 9, wherein a current flowing between the source electrode and the drain electrode of the transistor is measured.

13. The method according to claim 9, wherein the measuring step is conducted while applying an electric potential of V between the source and the drain.
